(19) United States
(12) Patent Application Publication
(10) Pub. No.: US 2008/0187508 A1
(43) Pub. Date: Aug. 7, 2008

(54) Treatment of Golmerular Basement Membrane Disease Involving Matrix Metalloproteinase-12

(75) Inventor: Dominic E. Cosgrove, Omaha, NE (US)

Correspondence Address: MUETING, RAASCH & GEBHARDT, P.A., P.O. BOX 581336, MINNEAPOLIS, MN, 55458-1336, US

(73) Assignee: BOYS TOWN NATIONAL RESEARCH HOSPITAL, Omaha, NE (US)

(21) Appl. No.: 11/662,122

(22) PCT Filed: Sep. 8, 2005
(86) PCT No.: PCT/US2005/031751

§ 371 Date: Sep. 21, 2007

Related U.S. Application Data
(60) Provisional application No. 60/607,907, filed on Sep. 8, 2004.
(60) Provisional application No. 60/686,148, filed on Jun. 1, 2005.

(51) Int. Cl. A61K 31/74; A61K 39/395; A61K 31/165; A61M 1/14; A61K 31/7088; A61P 13/12

(52) U.S. Cl. 424/78.08; 424/130.1; 514/44; 514/618; 604/5.04

(57) ABSTRACT

Methods for treating glomerular basement membrane disease such as Alport syndrome involving matrix metalloproteinase-12 are disclosed. Treatment may be affected, for example, by administering matrix metalloproteinase-12 inhibitors, by administering CCR2 receptor inhibitors, or by administering MCP-1 inhibitors. Matrix metalloproteinase formation is affected by the CCR2 receptor, which is stimulated by the MCP-1 chemokine.

### CONTINUING APPLICATION DATA

This application claims the benefit of U.S. Provisional Application Ser. No. 60/607,907, filed Sep. 8, 2004, and U.S. Provisional Application Ser. No. 60/686,148, filed Jun. 1, 2005, which are incorporated by reference herein.

### GOVERNMENT FUNDING

The present invention was made with government support under Grant Nos. R01DK55000 and R01DC04844, awarded by the National Institutes of Health. The Government may have certain rights in this invention.

### BACKGROUND

Alport syndrome is a glomerular basement membrane (GBM) disease caused by mutations in type IV collagen genes. A glomerulus is a tiny cluster of looping blood vessels within the bowman's capsule of the kidney. Glomerulus derives from the Greek word for “filter”, and the glomeruli, of which there are approximately 1 million, play an important role in blood filtration by the kidney. Within the glomerulus is the glomerular capillary wall. The glomerular capillary wall is unusual in that it has three layers: a fenesrated endothelium, the glomerular basement membrane, and the foot processes of glomerular epithelial cells. A unique irregular thickening and thinning of the GBM characterizes the progressive glomerular pathology. The metabolic imbalances responsible for these GBM irregularities are not known.

Alport syndrome has become a leading model for genetic disorders affecting basement membranes. The gene frequency is about 1 in 5000 people, making it among the more prevalent of known genetic disorders (Atkin et al. (1988) Diseases of the Kidney, 4th ed., Chap. 19, Little Brown, Boston, pp. 617-641) and Pescucci et al. (2003) J. Nephrol. 16, 314-316). It has been determined that X-linked Alport syndrome is caused by any of a series of mutations in the collagen 4A5 gene (Barker et al. (1990) Sci. 348, 1224-1227). At least 60 different mutations in the gene have been identified in families carrying the disease thus far (Tryggvason et al. (1993) Kidney Int. 43, 38-44 and Antignac et al. (1994) Am. Soc. Clin. Invest. 93, 1195-1207). The autosomal form of Alport syndrome, which displays the same range of phenotypes as the X-linked form of the disease, is due to mutations in either basement membrane collagen gene 4A3 or 4A4 (Lemmink et al. (1994) Hum. Mol. Gen. 3, 1269-1273 and Mochizuki et al. (1994) Nature Genet. 8, 77-81). Alport syndrome is characterized by a juvenile onset of proteinuria. The protein in the urinary space of the glomerulus precedes changes in glomerular cell types including ciliated podocytes and expansion of the mesangium. These changes culminate in accumulation of extracellular matrix, resulting in focal and segmental glomerulonephritis. The glomeruli eventually become fibrotic, resulting in a decreased capacity of the kidney to filter the blood. This ultimately results in a fatal uremia. Current therapy is limited to transplantation, with a high risk of rejection due to immune reaction against the type IV collagen chains in the transplanted organ.

A hallmark and unique characteristic of Alport glomerular disease is an irregular thickening, thinning, and splitting of the glomerular basement membrane (Kashtan et al. (1999) Medicine 78, 338-360). The progressive GBM damage is associated with podocyte foot process effacement. The mechanism underlying this phenotype is unknown, however it has been suggested that thickened regions might represent areas of matrix deposition (Cosgrove et al. (1996) Genes Dev. 10, 2981-2992 and Cosgrove et al. (2000) Am. J. Pathol. 157, 1649-1659; Abrahamson et al. (2003) Kidney Int. 63, 826-834). Alternatively, it has been shown that type IV collagen matrix from Alport kidneys is more susceptible to endoproteolytic cleavage than that from normal kidneys (Kalluri et al. (1997) J. Clin. Invest. 99, 2470-2478). This is presumably due to a significant reduction of interchain disulfide crosslinks resulting from differences in collagen chain composition (Gunwar et al. (1998) J. Biol. Chem. 273, 8767-8775).

Alport syndrome is currently treated by dialysis and transplant. However, transplantation often results in an autoimmune reaction against the type IV collagen. Study of the molecular processes underlying Alport renal disease has been significantly enhanced by the development of animal model systems, resulting in the evolution of potential treatment modalities that are at varying stages of development. Ramipril, an ACE inhibitor currently in the field, doubles the lifespan of Alport mice (Gross et al. (2003) Kidney Int. 63, 438-446) and is currently under consideration for human clinical trials. Neutralization of integrin α1β1 is also doubles lifespan in the mouse model (Cosgrove et al. (2000) Am. J. Pathol. 157, 1649-1659), and a therapeutic approach involving neutralizing antibodies is entering phase II clinical trials. Gene therapy is also being developed for testing in animal models (Heikkila et. al. (2001) Gene Ther. 8, 882-890). However, at present, Alport syndrome remains a disease with no powerful or reliable treatment options.

### SUMMARY

In one aspect, the present invention provides a method for treating glomerular basement membrane disease in a subject that includes administering a matrix metalloproteinase-12 (MMP-12) inhibitor to the subject. In a further aspect, the present invention also provides a method for treating Alport syndrome in a subject that includes administering a matrix metalloproteinase-12 inhibitor to the subject. Administering a matrix metalloproteinase-12 inhibitor can also be used for a method of treating glomerular disease associated with Alport syndrome, a method for decreasing the irregularity of the width of the glomerular basement membrane associated with Alport syndrome, and a method for decreasing the degradation of extracellular matrix in the glomerular basement membrane. In one embodiment of the methods for using matrix metalloprotinease-12 inhibitors described above, administering the matrix metalloproteinase-12 inhibitor decreases matrix metalloproteinase-12 activity in glomerular podocytes.

In a further embodiment of the methods for using matrix metalloproteinase-12 inhibitors, the matrix metalloproteinase-12 inhibitor may be a non-peptidic inhibitor. A non-peptidic inhibitor may be an arylsulfonamide substituted hydroxamic acid derivative in a further embodiment. In yet another embodiment, the arylsulfonamide-substituted hydroxamic acid is MMI-270. Other non-peptidic inhibitors that may be used in further embodiments include thiophene amino acid derivatives, fluorothiophene derivatives, and 1-carboxymethyl-2-oxo-azepan derivatives. In further embodiments, the matrix metalloproteinase-12 inhibitor may be a polypeptide such as an antibody, or an oligonucleotide.

In an additional aspect, the present invention provides a method for treating glomerular basement membrane disease in a subject that includes administering a CC chemokine receptor 2 (CCR2) receptor inhibitor to the subject. In a further aspect, the present invention also provides a method for treating Alport syndrome in a subject that includes administering a CCR2 receptor inhibitor to the subject. Administering a CCR2 receptor inhibitor can also be used for a method of treating glomerular disease associated with Alport syndrome, a method for decreasing the irregularity of the width of the glomerular basement membrane associated with Alport syndrome, and a method for decreasing the degradation of extracellular matrix in the glomerular basement membrane. In one embodiment of the methods for using CCR2 receptor inhibitors described above, administering the CCR2 receptor inhibitor decreases matrix metalloproteinase-12 activity in glomerular podocytes.

In embodiments using a CCR2 receptor inhibitor as described above, the CCR2 receptor inhibitor may be a non-peptidic inhibitor. In a further embodiment, the non-peptidic small molecular inhibitor may be an organogermanium compound. In yet a further embodiment, the organogermanium compound is 3-oxygemylpropinic acid polymer. In further embodiments, the CCR2 receptor inhibitor may be a polypeptide such as an antibody, or an oligonucleotide.

In a further aspect, the present invention provides a method for treating glomerular basement membrane disease in a subject that includes administering a monocyte chemoattractant protein-1 (MCP-1) inhibitor to the subject. In yet another aspect, the present invention also provides a method for treating Alport syndrome in a subject that includes administering an MCP-1 inhibitor to the subject. Administering an MCP-1 inhibitor can also be used for a method of treating glomerular disease associated with Alport syndrome, a method for decreasing the irregularity of the width of the glomerular basement membrane associated with Alport syndrome, and a method for decreasing the degradation of extracellular matrix in the glomerular basement membrane. In one embodiment of the methods for using MCP-1 inhibitors described above, administering the MCP-1 inhibitor decreases matrix metalloproteinase-12 activity in glomerular podocytes.

In embodiments of the methods of using MCP-1 inhibitors described above, the MCP-1 inhibitor may be a non-peptidic inhibitor. In further embodiments, the MCP-1 inhibitor may be a polypeptide such as an antibody, or an oligonucleotide.

In additional embodiments of the methods of the invention, the inhibitor may be administered orally, intravenously, intramuscularly, intraperitoneally, and/or subcutaneously. The inhibitor may be a matrix metalloproteinase-12 inhibitor, a CCR2 receptor inhibitor, or an MCP-1 inhibitor.

Further embodiments of the methods of the invention include administering one or more additional treatment modalities. Additional treatment modalities may include kidney dialysis, administration of a corticosteroid, and administration of a non-steroidal anti-inflammatory drug (NSAID).

In a further aspect, the invention provides a method for treating glomerular basement membrane disease by decreasing matrix metalloproteinase-12 activity in a glomerulus of a subject by administering an MMP-12 inhibitor, a CCR2 receptor inhibitor, or a MCP-1 inhibitor, or a combination thereof. In one embodiment, the glomerular basement membrane disease is Alport syndrome. In a further embodiment, decreasing matrix metalloproteinase-12 activity in a glomerulus includes decreasing matrix metalloproteinase-12 activity in a glomerular podocyte.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or be naturally occurring. This term also includes polypeptides that have been modified or derivatized, such as by glycosylation, acetylation, phosphorylation, and the like.

The terms “oligonucleotides” and “oligonucleotide” as used herein are used interchangeably and refer to a polymer of nucleotides. The terms do not connote a specific length of a polymer of nucleotides. The oligonucleotide can be deoxyribonucleic acid (DNA) or ribonucleic acid (RNA). The oligonucleotides can be produced using recombinant techniques, chemical or enzymatic synthesis, or be naturally occurring. This term also includes polypeptides that have been modified or derivatized, such as by glycosylation, acetylation, phosphorylation, and the like.

Unless otherwise specified, “a,” “an,” “the,” and “at least one” are used interchangeably and mean one or more than one.

The terms “comprises” and variations thereof do not have a limiting meaning where these terms appear in the description and claims.

As used herein, the term “room temperature” refers to a temperature of about 20° C. to about 25° C. or about 22° C. to about 25° C.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 provides a bar graph showing the results of a real time PCR analysis of MMPs of glomerular RNA from 7-week-old normal and Alport mice. Asterisks denote statistically significant differences in specific MMP expression when comparing normal and Alport mice (p>0.005).

FIG. 2 provides illustrations of tissue sections showing that MMP 12 is upregulated in glomerular podocytes. Tissue sections from 7-week-old control and Alport mice were immunostained using antibodies specific for MMP-12 (panels A and B). Weak immunostaining is observed in normal mice (panel A) compared to robust immunostaining in Alport glomeruli (panel B), which appears to localize primarily to glomerular podocytes (arrowheads). In situ hybridization confirms induction of MMP-12 mRNA in glomerular podocytes of Alport mice Panel D), which appears to be absent in glomeruli from wild type mice (panel C). Panel F shows positive immunostaining for MMP-12 in human Alport glomeruli. Normal human glomeruli showed no detectable immunostaining (panel E).

FIG. 3 provides illustrations showing the results of dual immunofluorescence analysis of renal cortex from 7-week-old Alport and normal mice. Cryosections from normal and Alport mouse kidneys were immunostained with antibodies specific for MMP-12 (A and D) and CD11b (B and E). Dual immunostaining (C and F) illustrates that monocytes (red) are immuno-negative for MMP-12 (green) while glomeruli are immuno-positive for MMP-12.

FIG. 4 shows the results of a northern blot, which demonstrates that MMP-12 mRNA is induced as a function of Alport renal disease progression. Glomerular RNA from 7-week-old wild type, 7-week-old Alport, and 4-week-old Alport mice were analyzed by northern blot and probed with radiolabeled MMP-12 cDNA. The blot was stripped and re-probed with β-actin cDNA to control for RNA loading.

FIG. 5 provides immunostained tissue sections illustrating that treatment of Alport mice with a non-peptidic MMP inhibitor, MMI 270, arrests the progression of glomerular and tubulointerstitial pathogenesis associated with the disease. Kidneys were harvested and frozen sections were subjected to fluorescence immunostaining using antibodies specific for either fibronectin (panels A, B, C, and D) or type IV collagen α1 and α2 chains (panels E, F, G, and H). C: normal control mice (panels A and E); A: untreated Alport mice (panels B and F); A MMI 270: MMI270-treated Alport mice (C and G); A BAY 129566: Alport mice treated once daily with 4 mg of BAY 129566 by oral gavage in a 0.5% carboxymethly cellulose carrier. Note the remarkable improvement in both the glomerular and tubulointerstitial compartments of the MMI 270-treated Alport mice (C and G) relative to the BAY 129566-treated Alport mice (D and H), which showed no improvement relative to untreated Alport mice (B and F).

FIG. 6 shows the results of gel electrophoresis, demonstrating that treatment of Alport mice with MMI 270 arrests (and may reverse) progressive loss of glomerular function. Proteinuria was analyzed by gel electrophoresis. (A) One half microliter of urine from control mice (lane 2), MMI 270-treated normal control mice (lane 3), MMI 270-treated Alport mice (injected twice daily with 50 μg/g of NMI 270) (lane 4), and untreated Alport mice (lane 5) was fractionated on a 10% PAGE gel, stained with Coomassie blue, and destained. Molecular weight markers are shown in lane 1. (B) Progression of proteinuria appears to be arrested in Alport mice treated with MMI-270 from 6 to 7 weeks of age. Urine was collected and analyzed as in (A). Lane 1, 6 week old control; lane 2, 6 week old Alport; lane 3, 7 week old control; lanes 4 and 5, 7 week old Alport; lanes 6 and 7, 7 week old Alport treated with MMI-270 from 6 to 7 weeks of age. Molecular weight markers are shown in lane 1.

FIG. 7 shows that treatment with MMI-270 from 3 to 7 weeks results in markedly improved glomerular basement membrane architecture. Panel A, control mouse, panel B, Alport mouse, panel C, Alport mouse treated from 3 to 7 weeks with MMI-270.

FIG. 8 provides illustrations that shown that immuno-gold localization of type IV collagen reveals evidence for focal degradation in thickened regions of the GBM. Areas of the capillary loop that were structurally normal displayed a regular distribution of gold particles along the lamina densa (panel A). Areas where focal thickening of the GBM was observed showed evidence of focal degradation of the collagen network (panel B). Arrows denote evidence of collagen network splitting, where gold deposition is primarily on the epithelial or endothelial aspect of the GBM. There was no significant increase in gold particles per unit length of the GBM, suggesting that collagen is not accumulating in the thickened regions.

FIG. 9 shows the results of blot analysis that indicates that expression of both MCP-1 and CCR2 are induced in glomeruli from Alport mice relative to wild type mice. Panels A and B. RNA from isolated glomeruli (panel A lanes 3 and 4; panel B, lanes 1 and 2) or cultured glomerular podocytes (panel A, lanes 1 and 2) was amplified using primers specific for CCR2 (Panel A) or MCP1 (Panel B). GAPDH was amplified as a control. A=Alport; C=wild type; D=DKO. Panel C shows western blot analysis of protein from cultured mesangial cells (MC), cultured podocytes (Podo), or isolated glomeruli (glom) from normal (Wt) and Alport (Alp) mice probed with antibodies specific for CCR2.

FIG. 10 provides tissue sections showing that CCR2 mRNA is induced in glomerular podocytes. In situ hybridization analysis was performed on kidney sections from 7-week-old wild type (A and B) and Alport (C and D) mice using either sense (A and C) or antisense (B and D) riboprobes specific for the CCR2 transcript. Arrowheads denote representative glomerular podocytes.

FIG. 11 shows that treatment of Alport mice with propagermanium knocks down elevated MMP-12 expression and restores normal GBM architecture in Alport glomeruli. Panel I. Asterisks denote statistically significant differences in specific MMP expression when comparing normal and Alport mice (p>0.005). Note that only MMP-12 expression was affected by administration of propagermanium. Panel 11. Representative capillary loops from Alport mice treated with vehicle alone (panel A and C) or with propagermanium (panels B and D) were analyzed by transmission electron microscopy, restoration of uniform GBM thickness in the treated mice is observed along with reconstitution of normal podocyte foot processes and restoration of the slit diaphragms (figure D, arrow). Panels A and B, Bar=1 μm; panels C and D, Bar=50 nm.

FIG. 12 shows the chemical structure of MMI270.

FIG. 13 shows the amino acid sequence for human matrix metalloproteinase-12 proenzyme; Genbank Accession Number NP 002417.1 (SEQ ID NO:17).

FIG. 14 shows the amino acid sequence for isoform A of the human CCR2 receptor, Genbank Accession Number NP 000638 (SEQ ID NO:19).

FIG. 15 shows the amino acid sequence for human macrophage chemoattractant protein-1, Genbank Accession Number AAP35993 (SEQ ID NO:20).

### DETAILED DESCRIPTION OF ILLUSTRATIVE EMBODIMENTS

The present invention involves therapeutic strategies for the treatment of glomerular basement membrane disease. Glomerular basement membrane disease is a disease or disorder that impairs the proper functioning of the glomerulus, which is a capillary network within the bowman's capsule of the kidney.

Remodeling of the extracellular matrix (ECM) is an important physiologic feature of the normal growth and development, and a number of diseases have been associated with an imbalance of ECM synthesis and degradation (Arthur, M. J., Digestion (1989), 59, 376-380). Homeostatic ECM turnover is a delicately balanced system of coupled biosynthetic and degradative processes. The matrix metalloproteinase (MMP) family consists of over 25 members that collectively can degrade all components of the ECM. MMP activity is associated with several normal processes of tissue remodeling. Dysregulation of the MMPs may contribute to disease processes. The control and regulation of ECM degradation has been shown to be complex, and knowledge of the system in Alport syndrome is rudimentary. Preliminary evidence implicates a role for MMPs in renal pathogenesis associated with Alport syndrome (Rao et. al. (2003) Kidney Int. 63, 1736-1748) and Rodgers et al. (2003) Kidney Int. 63, 1338-55).

### Matrix Metalloproteinase-12

Matrix metalloproteinase (MMP) enzymes are major physiological regulators of ECM degradation in the glomerulus (Woessner, J. F. Jr., (1991) FASEB 5, 2145-2154). Changes in MMP expression or activity may result in altered ECM turnover, which may lead to glomerular scarring and a decrease in renal function. Many forms of glomerular disease are characterized by a change in cellularity, which may affect ECM composition and turnover. MMP enzymes have been shown to influence the behavior of glomerular cells, and have been implicated in a number of forms of glomerular disease, (Lenz et al. (2000) J. Am. Soc. Nephrol. 11, 574-581).

The matrix metalloproteinase family is a large family of zinc-dependent matrix-degrading enzymes, which include interstitial collagenases, stromelysins, gelatinases, elastases, and membrane-type RXKR containing MMP. The matrix metalloproteinase family includes, but is not limited to, fibroblast collagenase (MMP-1), gelatinase-A (MMP-2), stromelysin-1 (MMP-3), matrilysin (MMP-7), collagenase-2 (MMP-8), gelatinase-B (MMP-9), matrix metalloproteinase-10 (MMP-10), stromelysin-3 (MMP-11), macrophage metalloelastase (MMP-12), human collagenase-3 (MMP13), and membrane type I-matrix metalloproteinase (referred to as MT1-MMP or MMP-14).

The present invention relates to therapeutic strategies for the treatment glomerular disease. In one embodiment, this is accomplished by decreasing the level of matrix metalloproteinase-12 (MMP-12) activity. MMP-12 was first identified as an elastolytic metalloproteinase secreted by inflammatory macrophages (Banda and Werb (1981) Biochem. J. 193, 589-605) and structurally defined by Shapiro et al. (Shapiro et al., (1992) J. Biol. Chem. 267, 4664-4671 and Shapiro et al., (1993) J. Biol. Chem. 268, 23824-23829), and is designated EC number 3.4.24.65. MMP-12 is generally categorized as a metalloelastase, and one of its substrates is elastin. Other substrates include fibronectin, laminin, plasminogen, and tissue factor pathway inhibitor. Accordingly, MMP-12 is also referred to as macrophage elastase. Most MMP's are secreted as inactive proproteins that are activated when cleaved by extracellular proteinases. It is thought that the MMP-12 propeptide cleaved at both ends to yield the active enzyme. MMP-12 degrades soluble and insoluble elastin.

MMP-12 (matrix metalloproteinase-12) is a potent protease with broad matrix substrate specificity that has been associated with macrophages and lung disease (Gronski et al. (1997) J. Biol. Chem. 272, 12189-12194). To date, expression of MMP-12 has only been demonstrated in macrophages (Vos et al., J. Neuroimmunol. 2003; 38, 106-114) and Kaneko et. al. (2003) J. Immunol. 170, 3377-3385), hypertrophic osteoclasts (Hou et. al. (2004) Bone 34, 37-47), vascular smooth muscle cells (Wu et al. (2003) Genes Cells 8, 225-234), and some cancer cells (Ding et al., 2002, Oncology 63, 378-384; Zucker et al. (2004) Cancer Metastasis Rev. 23, 101-117).

The determinants of the substrate specificity of MMP-12, based on analysis of its crystal structure, have been described (Lang et al. (2001) J. Mol. Biol. 28, 731-742). Crystal structure analysis revealed an overall fold similar to that of other MMPs. However, an S-shaped double loop connecting strands III and IV is fixed closer to the beta-sheet and projects its H is 172 side-chain further into the hydrophobic active-site cleft, defining the S3 and the S1-pockets and separating them from each other to a larger extent than what is observed in other MMPs. The active-site cleft of MMP-12 is well equipped to bind and efficiently cleave the Ala-Met-Phe-Leu-Glu-Ala sequence (SEQ ID NO: B). However, MMP-12 appears to have broad substrate specificity, and is able to cleave sites within a large variety of substrates. (Gronski et al. (1997) J. Biol. Chem. 272, 12189-12194).

Matrix metalloproteinase-12 activity can be determined using various methods by those skilled in the art. For example, matrix metalloproteinase-12 levels may be determined in a tissue sample obtained by a biopsy. Generally, matrix metalloproteinases are assayed using synthetic quenched fluorescent substrates. For example, a specific fluorescent assay kit for MMP-12 using a quenched fluorescent peptide is available. This fluorescent assay kit is referred to as the AK-403 QUANTIZYME assay system, and is available from BIOMOL research laboratories (Plymouth Meeting, Pa.).

### Treatment Of Glomerular Basement Membrane Disease

The invention provides a method for treating glomerular basement membrane disease (e.g., Alport syndrome) in a subject. The subject is preferably a mammal, such as a domesticated farm animal (e.g., cow, horse, pig) or pet (e.g., dog, cat). More preferably, the subject is a human. Gomerular basement membrane (GBM) disease is distinguished from other glomerular disease by pathology that is present within the basement membrane itself. GBM disease generally results in hematuria and proteinuria that can be detected by techniques known to those skilled in the art.

As noted earlier, the GBM is one of three layers present in the glomerular capillary wall. The structure of the GBM is described by Deen et al. (Deen et al. (2001) Am. J. Physiol. Renal. Physiol. 281, F579-596). The GBM is a gel-like material that is 90-93% water by volume. Structural integrity is conferred by a heteropolymeric network of type IV collagen, laminin, fibronectin, entactin, and heparan sulfate proteoglycan. Collagen IV forms an interconnected network of fibers within the GBM, to which other matrix components are attached. Laminin is thought to play an important role in the structural integrity of the GBM and in its interactions with the cellular layers of the glomerular capillary wall. The sulfated glycoprotein entactin, or nidogen, binds to collagen IV, heparan sulfate proteoglycan, and laminin and may play an important role in linking GBM components to one another. Similarly, fibronectin binds to laminin, collagen IV, and heparan sulfate proteoglycan, suggesting that it too may have a role in linking GBM constituents together. Heparan sulfate proteoglycan has been shown to comprise 1% of the dry weight of the GBM. Glomerular basement membrane disease occurs when the GBM loses its capacity to properly function as semi-permeable barrier.

Alport syndrome is an X-linked genetic disorder that results in GBM disease caused by mutations in either of the basement membrane collagen genes 4A3 or 4A4. Alport syndrome is also known as hereditary nephritis, hemorrhagic familial nephritis, and hereditary deafness and nephropathy. One of the first symptoms of Alport's syndrome is usually hematuria, or blood in the urine. Tests also may reveal high levels of protein and white blood cells in the urine and waste products such as urea in the blood (uremia). Other symptoms may include hearing loss, particularly sounds at high frequencies; vision problems, such as cataracts, involuntary eye movements, and abnormalities of the cornea; nerve problems, such as polyneuropathy; skin problems; and low blood platelet counts that can compromise blood clotting. Patients with Alport syndrome may also develop nephrotic syndrome, which can cause high protein levels in the urine, low levels of a protein called albumin in the blood, and swelling, usually in the legs and/or abdomen. Nephrotic syndrome is caused by damage to the glomeruli. The structure of the glomeruli prevents most protein from getting filtered through into the urine. Normally, a healthy individual loses less than 150 mg of protein in the urine over a 24-hour period. However, in nephrotic-range proteinuria, the urination of more than 3.5 grams of protein during a 24-hour period, or 25 times the normal amount, may be observed.

Alport syndrome is clinically diagnosed based on an irregular thickening and thinning of the width of the renal GBM. Example 1, herein, demonstrates that MMP-12 mRNA and protein expression is markedly induced in glomeruli in an autosomal Alport mouse model.

Accordingly, in one aspect, the present invention uses the newly revealed understanding of the role of MMP-12 in glomerular basement membrane disease such as Alport syndrome to provide novel methods of treating glomerular basement membrane disease. Data indicates that MMP-12 induction lead to degradation of the glomerular basement membrane. Accordingly, inhibition of MMP-12 activity, in embodiments of the invention, may prevent degradation of the glomerular basement membrane. MMP-12 activity, in turn, may be regulated by CCR2 receptor activity. Further, CCR2 receptor activity may be influenced by the activity of the chemokine MCP-1, which stimulates the CCR2 receptor.

In one aspect, the present invention treats glomerular basement membrane disease (e.g., Alport syndrome) by decreasing matrix metalloproteinase-12 activity. Matrix metalloproteinase-12, as defined herein, is a polypeptide including an amino acid sequence with at least 90% identity, and more preferably 95% identity, to the polypeptide sequence of a characterized matrix metalloproteinase-12 enzyme that retains activity, as defined herein. Polypeptide sequences can be readily identified by those skilled in the art. For example, polypeptide sequences can be identified using mass spectrometry, Edman degradation, or prediction from oligonucleotide sequence. In a further embodiment, matrix metalloproteinase-12 is the enzyme, and substantially similar polypeptides, described by Gronski et al. (Gronski et al., J. Biol. Chem. (1997) 272, 12189-12194) or Shapiro et al. (Shapiro et al., (1993) J. Biol. Chem. 268, 23824-23829).

Inclusion of polypeptides with an amino acid sequence having at least 90% identity, and more preferably 95% identity, to the polypeptide sequence of a characterized matrix metalloproteinase-12 enzyme is intended to cover closely related forms of the enzyme, such as those that include minor mutations or other changes, but retain enzymatic activity. The similarity is referred to as structural similarity, and is generally determined by aligning the residues of a candidate polypeptide with the sequence of interest. For example, with MMP-12, a candidate MMP-12 enzyme amino acid sequence is aligned with a known sequence of MMP-12 to optimize the number of identical amino acids along the lengths of their sequences. Gaps in either or both sequences are permitted in making the alignment in order to optimize the number of identical amino acid sequences, but the amino acids in each sequence should remain in their proper order.

Preferably, two amino acid sequences are compared using the Blastp program of the BLAST 2 search algorithm, as described by Tatusova, et al. (FEMS Microbiol. Lett, 174:247-250 (1999)), and available at http://www.ncbi.nlm.nih.gov/blast/bl2seq/bl2.html. Preferably, the default values for all BLAST 2 search parameters are used, including matrix=BLOSUM62; open gap penalty=11, extension gap penalty=1, gap x_dropoff=50, expect=10, wordsize=3, and optionally, filter on. In the comparison of two amino acid sequences using the BLAST search algorithm, structural similarity is referred to as “identities.”

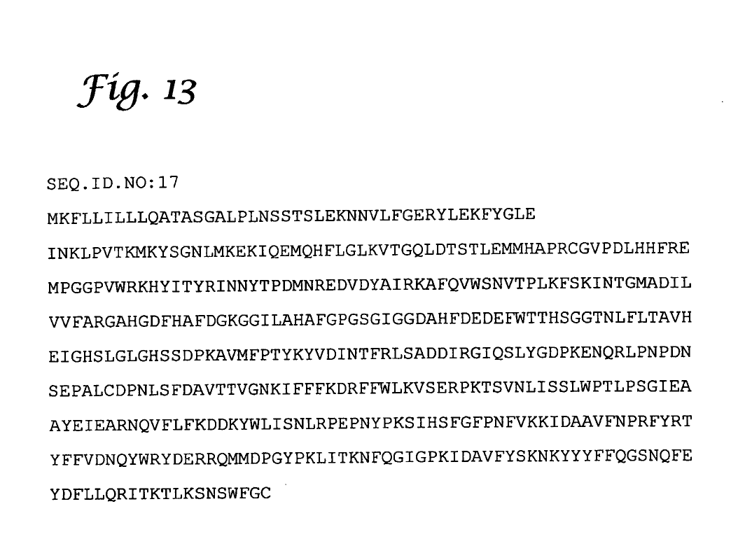

For example, SEQ ID NO: 17, shown in FIG. 13, provides the amino acid sequence for a proenzyme form of matrix metalloproteinase-12, determined by Shapiro et al., J Biol. Chem. (1993) 268(32), 23824-9, and assigned Genbank Accession Number NP 002417.1. The proenzyme version includes the amino acid sequence of matrix metalloproteinase-12, while also including additional amino acids that are cleaved to form the active form of matrix metalloproteinase-12 (Gronski et al., J. Biol. Chem. (1997) 272, 12189-12194).

More particularly, the activity of MMP-12 refers to the ability of MMP-12 to function as a protease, and more specifically as an elastase. The ability to function as an elastase provides MMP-12 with the ability to cleave a number of polypeptide substrates. For example, MMP-12 has been shown to have the capacity to cleave polypeptides containing SEQ ID NO:18. Thus, the phrase “decreasing the level of activity”, as used herein, refers to decreasing the level of activity of MMP-12 in a subject relative to the MMP-12 activity that is present in the subject when an inhibitor (e.g., an MMP-12 inhibitor, a CCR2 receptor inhibitor, or an MCP-1 inhibitor) is not administered. Activity may be decreased by at least 50%, and may be decreased by at least 75% or at least 90% in further embodiments of the invention.

In one aspect, the present invention treats Alport syndrome by directly decreasing the activity of MMP-12. In another aspect, the present invention treats Alport syndrome by indirectly decreasing the activity of MMP-12 by decreasing its formation. MMP-12 is highly regulated by cytokines. Known pathways for MMP-12 activation include GM-CSF, MCP-1, and PDGF-BB (Wu et al. (2000) Biochem. Biophys. Res. Commun. 269, 808-815; Feinberg et al. (2000) J. of Bio. Chem. 275, 25766-25773; Jost et al. (2003) FASEB J. 17, 2281-2283). All three of these cytokines have been shown to be induced in various glomerular diseases as well as in mesangial cell culture systems. However, the results provided in Example 1 show that the cellular mechanism of MMP-12 induction in glomerular podocytes is MCP-1 activation of the CCR2 receptor. Accordingly, embodiments of the present invention provide methods of treating Alport syndrome by decreasing activation of the CCR2 and/or decreasing the level of MCP-1, thereby decreasing CCR2 receptor stimulation.

Methods of the present invention thus provide a variety of avenues for treatment of GBM disease and/or Alport syndrome by use of inhibitors that, in some embodiments, decrease MMP-12 activity. For example, matrix metalloproteinase-12 activity may be decreased by administering a matrix metalloproteinase-12 inhibitor. Matrix metalloproteinase-12 activity may also be decreased by administering an inhibitor of the CCR2 receptor, thereby decreasing the formation of MMP-12. Matrix metalloproteinase-12 activity may also be decreased by administering an inhibitor of MCP-1. As MCP-1 activates the CCR2 receptor, inhibiting MCP-1 decreases the formation of MMP-12. Matrix metalloproteainse-12 activity may also be decreased through a combination of the methods described above. For example, GBM disease and/or Alport syndrome may be treated by administering both an MMP-12 inhibitor and a CCR2 receptor inhibitor. Combined administration of multiple methods of treatment may result in synergistic effects in treatment of GBM disease and/or Alport syndrome in a subject.

### Treatment by Administration of MMP-12 Inhibitory

In one embodiment, the present invention provides a method for treating glomerular basement membrane disease such as Alport syndrome by administering to a subject a matrix metalloproteinase-12 inhibitor. A matrix metalloproteinase-12 inhibitor (MMP-12 inhibitor), as defined herein, is an agent that acts upon matrix metalloproteinase-12 or inhibits its biosynthesis to result in decreased matrix metalloproteinease-12 activity. A matrix metalloproteinase-12 inhibitor may also be referred to herein as an inhibitor of matrix metalloproteinase-12. In one aspect, the matrix metalloproteinease-12 is a proteinase inhibitor that has an inhibitory effect on matrix metalloproteinase-12.

The matrix metalloproteinase-12 inhibitor need not be specific for only MMP-12, and may have an effect on other enzymes, though embodiments of the invention may use specific MMP-12 inhibitors. The MMP-12 inhibitor also need not act on catalytic site of MMP-12, but rather may inhibit MMP-12 in various other ways, e.g., by sterically hindering the active site, distorting the enzyme structure, or preventing access to necessary ions or cofactors.

The MMP-12 inhibitor may be administered systemically, or it may be administered preferentially to the kidney. Preferential administration to the kidney may be achieved by direct delivery to the kidney, pharmacokinetic means, or by use of targeting agents specific for the kidney. In one embodiment, administration of the MMP-12 inhibitor decreases of the level of MMP-12 activity in glomerular podocytes.

A variety of types of agents may be used as MMP-12 inhibitors. For example, the MMP-12 inhibitor may be a non-peptidic inhibitor. Matrix metalloproteinase-12 inhibitors include MMP-12 inhibitors designed using any of the various structure-based design approaches routinely used in the pharmaceutical and medicinal chemistry fields (as reviewed by Matter and Schudok (Curr Opin Drug Discov Devel. 2004 July; 7(4):513-35)). Examples of such non-peptidic MMP-12 inhibitors include, for example, hydroxamic acid derivatives, such as the arylsulfonamido-substituted hydroxamic acids and salts thereof presented in U.S. Pat. Nos. 5,552,419 and 5,672,615 and the sulfonylamino acid and sulfonylamino hydroxmic acid derivatives presented in U.S. Pat. Nos. 6,277,987 and 6,410,580. Other examples of non-peptidic MMP-12 inhibitors include thiophene amino acid derivatives, as described by Compere et al. in U.S. Patent Application Publication No. 2005/0014816, fluorothiophene derivatives described by Compere et al. in U.S. Patent Application Publication No. 2005/0014817, and 1-carboxymethyl-2-oxo-azepan derivatives described by Warshawsky et al. in U.S. Pat. No. 6,770,640.

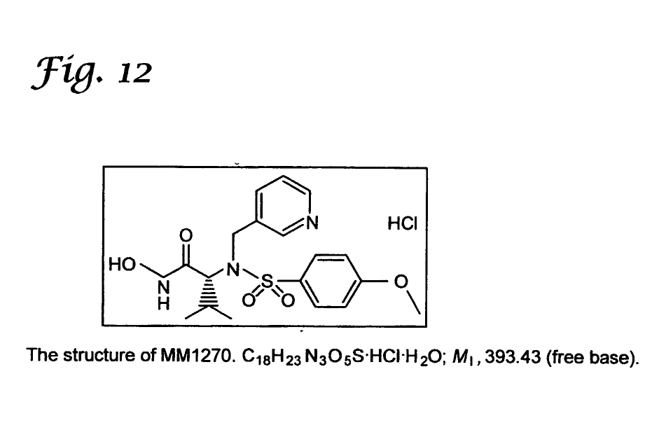

In one embodiment, the MMP-12 inhibitor is MMI-270, N-hydroxy-2(R)-[(4-methoxysulfonyl)(3-picolyl)amino]-3-methylbutaneamide hydrochloride monohydrate, also known as CGS 27023A (MacPherson et al., J Med. Chem. 1997 Aug. 1; 40(16):2525-32). MMI-270, also referred to herein as “MMI270,” is shown in FIG. 12 and is a novel synthetic hydroxamic acid derivative able to competitively bind the Zn²⁺ ion in the active site of a wide range of MMPs, inhibiting their activity at nM concentrations in vitro. The oral administration of the MMI-270 may follow the procedures used in the Phase I and pharmacological studies reported by Levitt et al. (Clin Cancer Res. 2001 July; 7(7):1912-22).

In another embodiment, The MMP-12 inhibitor may be a peptide. For example, the MMP-12 inhibitor may be an antibody that specifically binds to MMP-12. As used herein, the phrase “specifically binds” and other permutations of the phrase refers to an antibody that will, under appropriate conditions, preferentially interact with a desired antigen rather than a different antigen. MMP-12, a peptide that includes numerous amino acids, provides a number of antigenic sites that can be used to generate an antibody that specifically binds to MMP-12. Antibody to MMP-12 can act as an MMP-12 inhibitor in various ways, e.g., by blocking the MMP-12 active site or causing MMP-12 to be removed by the immune system.

Antibodies are produced by B cells and are a type of globulin protein called immunoglobulins. There are five major classes of immunoglobulins, designated IgA, IgD, IgE, IgG, and IgM. Antibody molecules are able to chemically recognize surface portions, or epitopes, of large molecules that act as antigens, such as nucleic acids, proteins, and polysaccharides. Generally, an antibody that binds to a polypeptide recognizes an epitope on the polypeptide that includes about 6 amino acids, although as few as 2 amino acids may be effective in some circumstances. Immunoglobulin G (IgG) antibodies consist of four polypeptide chains, two identical heavy chains and two identical light chains. Antibody molecules of a particular class have a similar overall structure, except for certain small segments that varying in amino acid sequence, accounting for the specificity of the antibodies for particular antigens.

Accordingly, antigens present on MMP-12 can be used to produce antibodies, including vertebrate antibodies, hybrid antibodies, chimeric antibodies, humanized antibodies, altered antibodies, univalent antibodies, monoclonal and polyclonal antibodies, Fab proteins and single domain antibodies. If desired, MMP-12 can be modified by covalently linking them to an immunogenic carrier, such as keyhole limpet hemocyanin (KLH), bovine serum albumin, ovalbumin, mouse serum albumin, rabbit serum albumin, and the like.

If polyclonal antibodies are desired, a selected animal (e.g., mouse, rabbit, goat, horse or bird, such as chicken) is immunized with an antigen from MMP-12. Techniques for producing and processing polyclonal antisera are known in the art (see for example, Mayer and Walker eds. Immunochemical Methods in Cell and Molecular Biology (Academic Press, London) (1987), Coligan, et al., Unit 9, Current Protocols in Immunology, Wiley Interscience (1991), Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, section 2.4.1 (1992)).

In another aspect, the MMP-12 inhibitors are monoclonal antibodies directed against MMP-12. Monoclonal antibodies can be readily produced by one skilled in the art. The general methodology for making monoclonal antibodies by hybridomas is well known. Immortal antibody-producing cell lines can be created by cell fusion, and also by other techniques such as direct transformation of B lymphocyte cells with oncogenic DNA, or transfection with Epstein-Barr virus (See Monoclonal Antibody Production. Committee on Methods of Producing Monoclonal Antibodies, Institute for Laboratory Animal Research, National Research Council; The National Academies Press; (1999), Kohler & Milstein, Nature, 256:495 (1975); Coligan et al., sections 2.5.1-2.6.7; and Harlow et al., Antibodies: A Laboratory Manual, page 726 (Cold Spring Harbor Pub. 1988)).

In another aspect of the invention, MMP-12 inhibitors include oligonucleotides that inhibit the formation of MMP-12 at the molecular genetic level. Such oligonucleotides can be designed in light of the oligonucleotide sequence for MMP-12, which can be readily found or determined by those skilled in the art. Sequences are also currently known for the MMP-12 gene in the mouse, rat, and rabbit. For example, MMP-12 inhibitors may include short interfering ribonucleic acids (siRNA) that have been designed to silence translation and/or transcription of MMP-12. siRNA designed to silence transcription of specific polypeptides can be readily prepared by those skilled in the art. For example, over 1,000 siRNA designed to silence transcription of specific polypeptides are available from AMBION, who will also custom design siRNA on demand. As an alternative to siRNA, antisense oligonucleotides can be used to block formation of MMP-12, and hence serve as MMP-12 inhibitors. Antisense oligonucleotides, typically 18 to 25 nucleotides in length, are designed to bind to a complementary sequence in a MMP-12 mRNA, preventing translation of the MMP-12 mRNA. Embodiments of the invention using antisense oligonucleotides as MMP-12 inhibitors may use antisense oligonucleotide morpholino or phosphorothioate derivatives that provide increased resistance to degradation by nucleases. Antisense oligonucleotides can be readily prepared by those skilled in the art and are available, for example, from Gene Tools, LLC.

### Treatment by Administration of CCR2 Receptor Inhibitors

The present invention also provides methods for treating glomerular basement membrane disease and/or Alport syndrome by administering CCR2 receptor inhibitors. Administration of CCR2 receptor inhibitors, in embodiments of the invention, decreases CCR2 receptor activity, which, in further embodiments, may decrease the level of matrix metalloproteinase-12 activity in a subject. A CCR2 receptor inhibitor, as defined herein, is an agent that acts upon the CCR2 receptor or inhibits its biosynthesis to result in a decrease in CCR2 receptor activity. A decrease in CCR2 receptor activity may occur in a variety of different ways. For example, CCR2 receptor activity may be decreased by lowering the number of CCR2 receptors, antagonizing existing receptors, modifying the receptors, suppressing signaling transmitted from the receptor, and/or decreasing the stimulus received from MCP-1.

The CCR2 receptor inhibitor may be administered systemically, or it may be administered preferentially to the kidney. Preferential administration to the kidney may be achieved by direct delivery to the kidney, pharmacokinetic means, or by use of targeting agents specific for the kidney. In one embodiment, administration of a CCR2 receptor inhibitor decreases of the level of MMP-12 activity in glomerular podocytes.

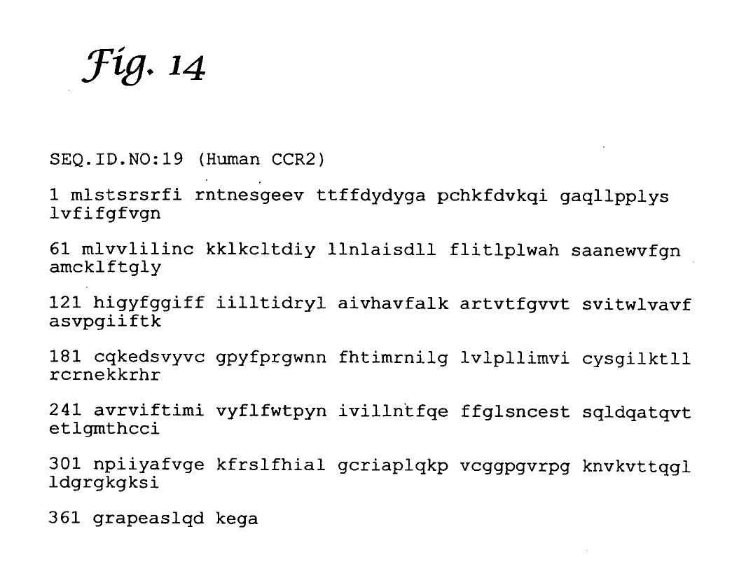

The CC chemokine receptor 2 (CCR2 receptor) is a receptor for monocyte chemoattractant protein-1 (MCP-1), a chemokine that mediates monocyte chemotaxis. The receptor mediates agonist-dependent calcium mobilization and inhibition of adenylyl cyclase. This gene encoding CCR2 is located in the chemokine receptor gene cluster region. Two alternatively spliced transcript variants are expressed by the gene. The first variant (A) encodes a cytoplasmic isoform. It is alternatively spliced in the coding region resulting in a frameshift and use of a downstream stop codon, compared to variant B. Isoform A, accession number NP 000638, has distinct C-terminus and is 14 amino acids longer than isoform B, and has the sequence shown in SEQ ID NO:19, and shown in FIG. 14. See Charo et al., Proc. Natl. Acad. Sci. U.S.A. (1994) 91, 2752-2756.

The CCR2 receptor, as defined herein, is a polypeptide including an amino acid sequence with at least 90% identity, and more preferably 95% identity, to the polypeptide sequence of a characterized CCR2 receptor that retains the ability to stimulate MMP-12 formation. Polypeptide sequences can be readily identified by those skilled in the art. For example, polypeptide sequences can be identified using mass spectrometry, Edman degradation, or prediction from oligonucleotide sequence. In a further embodiment, CCR2 is the receptor, and substantially similar polypeptides, as described by Charo et al. (Charo et al., Proc. Natl. Acad. Sci. U.S.A. (1994) 91, 2752-2756).

Inclusion of polypeptides with an amino acid sequence having at least 90% identity, and more preferably 95% identity, to the polypeptide sequence of a characterized CCR2 receptor is intended to cover closely related forms is intended to cover closely related forms of the receptor, such as those that include minor mutations or other changes, but retain the ability to stimulate MMP-12 formation. Sequence similarity may be determined as described herein, preferably using the Blastp program of the BLAST 2 search algorithm.

In one embodiment, the activation of the CCR2 receptor is decreased by administering to the subject a CCR2 receptor inhibitor. A CCR2 receptor inhibitor, as defined herein, is an agent that acts upon the CCR2 receptor or inhibits its biosynthesis to result in decreased CCR2 activity. A CCR2 receptor inhibitor may also be referred to herein as an inhibitor of the CCR2 receptor. The CCR2 receptor inhibitor need not be specific for only the CCR2 receptor, and may have an effect on other receptors, though embodiments of the invention may use specific CCR2 receptor inhibitors.

A variety of types of agents may be used as CCR2 receptor inhibitors. For example, the CCR2 receptor inhibitor may be a non-peptidic inhibitor. CCR2 receptor inhibitors include CCR2 receptor inhibitors designed using any of the various structure-based design approaches routinely used in the pharmaceutical and medicinal chemistry fields (as reviewed by Matter and Schudok (Curr Opin Drug Discov Devel. 2004 July; 7(4):513-35)). Examples of CCR2 receptor inhibitors include, for example, organogermanium compounds of the type disclosed in U.S. Pat. Nos. 5,532,272 and 5,621,003, pyrrolidinone and pyrrolidine-thiones disclosed in U.S. Pat. Nos. 6,936,633, and 3-cycloalkylaminopyrrolidine derivatives disclosed in U.S. Patent Application Publication No. 2005/0192302. A preferred organogermanium compound is the antagonist propagermanium acid polymer (i.e., 3-oxygermylpropionic acid).

Propagermanium inhibits CCR2 activity via targeting glycosylphosphatidylinositol-anchored proteins closely associated with CCR2 (Yokochi et al. (2001) J. Interferon Cytokine Res. 21, 389-398). Since CCR2 activation by MCP-1 is associated with acute and chronic inflammatory response mechanisms, animal studies to date utilizing propagermanium have focused on its anti-inflammatory activity, and include atheroschlerosis, renal fibrosis, and liver disease (Yokochi et al., 2001; Eto et al., 2003; Kitagawa et al.). The therapeutic potential has been focused on the pivotal role of CCR2 activation by MCP-1 in monocyte/lymphocyte recruitment to sites of local inflammation (Dambach et al. (2002) Hepatology 35, 1093-1103; Maus et al., (2002) Am. J. Respir. Crit. Care Med. 166, 268-273; Zernecke et. al. (2001) J. Immunol. 166, 5755-5762). This is the first report demonstrating a role for this system in a pathobiological system not involving monocyte/lymphocyte recruitment.

CCR2 receptor inhibitors may, in some embodiments of the invention, be peptides. For example, antibodies (including antibody fragments) that specifically bind to the CCR2 receptor can be used as CCR2 receptor inhibitors. In one embodiment, antibodies that are specific for the MCP-1 binding site on the CCR2 receptor may be used. However, antibodies specific for any portion of the CCR2 receptor that will reduce activity upon binding to or near the receptor may be used. These antibodies (including antibody fragments) include polyclonal, monoclonal, anti-idiotype, animal-derived, humanized and chimeric antibodies. Polyclonal and monoclonal antibodies may be prepared using the procedures described herein. For example, a monoclonal antibody useful as a CCR2 receptor inhibitor is described in U.S. Patent Application Publication No. 2002/0042370.

In another aspect of the invention, CCR2 receptor inhibitors include oligonucleotides that inhibit the formation of CCR2 at the molecular genetic level. Such oligonucleotides can be designed in light of the sequence for human CCR2 gene, described by Charo et al. (Proc. Natl. Acad. Sci. U.S.A. (1994) 91 (7), 2752-2756), and provided with accession number NM 000647. Sequences are also known for the CCR2 receptor gene in the mouse, pig, dog, and cow. For example, CCR2 receptor inhibitors may include short interfering ribonucleic acids (siRNA) that have been designed to silence translation and/or transcription of the CCR2 receptor. siRNA designed to silence transcription of specific polypeptides can be readily prepared by those skilled in the art. For example, over 1,000 siRNA designed to silence transcription of specific polypeptides are available from AMBION, who will also custom design siRNA on demand. As an alternative to siRNA, antisense oligonucleotides can be used to block formation of the CCR2 receptor, and hence serve as CCR2 receptor inhibitors. Antisense oligonucleotides, typically 18 to 25 nucleotides in length, are designed to bind to a complementary sequence in a CCR2 receptor mRNA, preventing translation of the CCR2 receptor mRNA. Embodiments of the invention using antisense oligonucleotides as CCR2 receptor inhibitors may use antisense oligonucleotide morpholino or phosphorothioate derivatives that provide increased resistance to degradation by nucleases. Antisense oligonucleotides can be readily prepared by those skilled in the art and are available, for example, from Gene Tools, LLC.

### Treatment by Administration of MCP-1 Inhibitors

The present invention also provides methods for treating glomerular basement membrane disease and/or Alport syndrome by administering MCP-1 inhibitors. In embodiments of the invention, administration of MCP-1 inhibitors decreases MCP-1 activity. A decrease in MCP-1 activity may lead, in some embodiments, to a decrease in CCR2 receptor activity, which in turn decreases the level of matrix metalloproteinase-12 activity in a subject. An MCP-1 inhibitor, as defined herein, is an agent that acts upon MCP-1 or inhibits its biosynthesis to result in a decrease in MCP-1 activity. A decrease in MCP-1 activity may occur in a variety of different ways. For example, MCP-1 activity may be decreased by decreasing the amount of MCP-1 available. The amount of MCP-1 may be decreased by prevention the biosynthesis of MCP-1 or by eliminating existing MCP-1. MCP-1 activity may also be decreased, for example, through partial degradation of MCP-1, blocking the active regions of MCP-1, or sequestering it to prevent it reaching the CCR2 receptor.

The MCP-1 inhibitor may be administered systemically, or it may be administered preferentially to the kidney. Preferential administration to the kidney may be achieved by direct delivery to the kidney, pharmacokinetic means, or by use of targeting agents specific for the kidney. In one embodiment, administration of a MCP-1 inhibitor decreases of the level of MMP-12 activity in glomerular podocytes.

MCP-1, as defined herein, is a polypeptide including an amino acid sequence with at least 90% identity, and more preferably 95% identity, to the polypeptide sequence of a characterized MCP-1 chemokine that retains the ability to stimulate the CCR2 receptor. Polypeptide sequences can be readily identified by those skilled in the art. For example, polypeptide sequences can be identified using mass spectrometry, Edman degradation, or prediction from oligonucleotide sequence. In a further embodiment, the MCP-1 chemokine is the polypeptide, and substantially similar polypeptides, described by Chang et al., Int. Immunol. (1989) 1, 388-397, or Yoshimura et al. (Yoshimura et al., Adv. Exp. Med. Biol. (1991) 305, 47-56).

Inclusion of polypeptides with an amino acid sequence having at least 90% identity, and more preferably 95% identity, to the polypeptide sequence of a characterized MCP-1 chemokine is intended to cover closely related forms is intended to cover closely related forms of MCP-1, such as those that include minor mutations or other changes, but retain the ability to stimulate MMP-12 formation. Sequence similarity may be determined as described herein, preferably using the Blastp program of the BLAST 2 search algorithm.

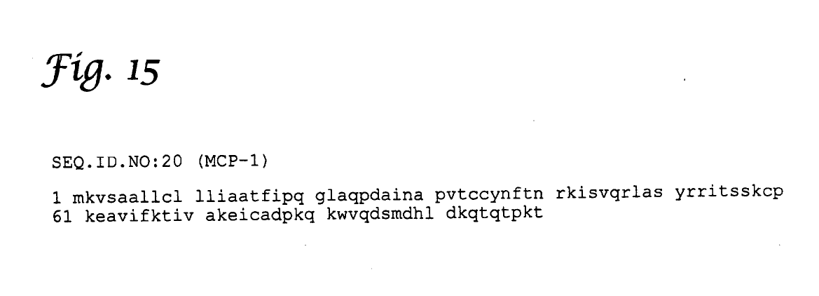

An example of a characterized form of MCP-1 is human MCP-1. The amino acid sequence of human MCP-1 is shown in FIG. 15, is represented by SEQ ID NO:20, and is assigned accession number AAP35993. MCP-1 is also referred to as the CCL2 ligand. Decreased activation of the CCR2 receptor by MCP-1 may be accomplished in various different ways. For example, MCP-1 formation may be decreased, or MCP-1 binding to the CCR2 receptor may be blocked. In one embodiment of the invention, MCP-1 activation of the CCR2 receptor is decreased by administering to the subject an MCP-1 inhibitor. An MCP-1 inhibitor, as defined herein, is an agent that acts upon MCP-1 or inhibits its biosynthesis to result in decreased CCR2 receptor activation by MCP-1.

A variety of types of agents may be used as MCP-1 inhibitors. For example, the MCP-1 inhibitor may be a non-peptidic inhibitor. See, for example, U.S. Pat. No. 6,809,113 and No. 6,737,435, which provide a number of compounds that function as MCP-1 antagonists. In a further embodiment, the MCP-1 inhibitor may be a peptide. For example, antibodies (including antibody fragments) that specifically bind to MCP-1 can be used as MCP-1 inhibitors. Antibodies may be monoclonal or polyclonal antibodies, and can be readily prepared by those skilled in the art, as described herein. MCP-1 may also be inhibited by gene therapy techniques by using oligonucleotides that inhibit the formation of CCR2 at the molecular genetic level. The amino acid sequence for the CCL2 ligand has been determined in humans, mice, cows, and dogs. Such oligonucleotides can be designed in light of the sequence for the human MCP-1 gene, described by Chang et al., Int. Immunol. (1989) 1, 388-397, and is available at the NCBI under accession number NM 002982.

### Additional Treatment Modalities

In the methods of the present invention, one or more additional treatment modalities may be used to supplement treatment of glomerular basement membrane disease (e.g. Alport syndrome) using inhibitors that may, in some embodiments, decrease the level of MMP-12 activity. A treatment modality is defined herein as therapeutic method or agent, such as surgery or chemotherapy, that involves the physical treatment of a disorder. Additional treatment modalities useful in the present invention may include, but are not limited to, kidney dialysis, administration of a corticosteroid, and/or administration of a non-steroidal anti-inflammatory drug (NSAID). Such additional treatment modalities may be administered before, after, and/or coincident with the administration of agents.

Kidney dialysis may include, for example, hemodialysis and peritoneal. dialysis. Hemodialysis uses a cellulose-membrane tube that is immersed in a large volume of fluid. Blood is pumped through this tubing, and then back into the patient's vein. The membrane has a molecular-weight cut-off that will allow most solutes in the blood to pass out of the tubing but retain the proteins and cells. Peritoneal dialysis, on the other hand, does not use an artificial membrane, but rather uses the lining of the patient's abdominal cavity, known as the peritoneum, as a dialysis membrane. Fluid is injected into the abdominal cavity, and solutions diffuse from the blood into this fluid. After several hours, the fluid is removed with a needle and replaced with new fluid.

Administration of corticosteroids and/or non-steroidal anti-inflammatory drugs represents an additional treatment modality. Corticosteroids include any one of several synthetic or naturally occurring substances with the general chemical structure of steroids that are used therapeutically to mimic or augment the effects of the naturally occurring corticosteroids, which are produced in the cortex of the adrenal gland. Examples of corticosteroids include prednisone, betamethasone, methylprednisolone acetate, hydrocortisone, and dexamethasone. Corticosteroids are effective as an additional treatment modality as they suppress the immune system and reduce inflammation within the kidney.

Non-steroidal anti-inflammatory drugs (NSAIDs) may also be used to reduce inflammation within the kidney as an additional treatment modality. NSAIDs include, for example, celecoxib, diclofenac, diflunisal, etodolac, fenoprofen, flurbirofen, ibuprofen, indomethacin, ketoprofen, ketorolac, mefenamic acid, meloxicam, nabumetone, naproxen, oxaprozin, piroxicam, sulindac, and tolmetin.

### Formulation and Administration of Agents

In the methods of the present invention, agents may be administered by one or more of the many routes utilized for the administration of a therapeutic agent to a subject. For example, an MMP-12 inhibitor may be administered orally, topically, intravenously, intramuscularly, intraperitoneally, and/or subcutaneously.

The methods of the invention include administering to a patient (i.e., a subject), preferably a mammal, and more preferably a human, an MMP-12 inhibitor (including an inhibitor of the CCR2 receptor) in an amount effective to produce the desired effect. An MMP-12 inhibitor (including an inhibitor of the CCR2 receptor) may be formulated for enternal administration (oral, rectal, etc.) or parenteral administration (injection, internal pump, etc.). The administration can be via direct injection into tissue, interarterial injection, intervenous injection, or other internal administration procedures, such as through the use of an implanted pump, or via contacting the composition with a mucous membrane in a carrier designed to facilitate transmission of the composition across the mucous membrane such as a suppository, eye drops, inhaler, or other similar administration method or via oral administration in the form of a syrup, a liquid, a pill, capsule, gel coated tablet, or other similar oral administration method. The active agents can be incorporated into an adhesive plaster, a patch, a gum, and the like, or it can be encapsulated or incorporated into a bio-erodible matrix for controlled release.

The carriers for internal administration can be any carriers commonly used to facilitate the internal administration of compositions such as plasma, sterile saline solution, IV solutions or the like. Carriers for administration through mucous membranes can be any well known in the art. Carriers for administration orally can be any carrier well known in the art.

The formulations may be conveniently presented in unit dosage form and may be prepared by any of the methods well known in the art of pharmacy. All methods may include the step of bringing the active agent into association with a carrier, which constitutes one or more accessory ingredients. In general, the formulations are prepared by uniformly and intimately bringing the active agent into association with a liquid carrier, a finely divided solid carrier, or both, and then, if necessary, shaping the product into the desired formulations.

Formulations suitable for parenteral administration conveniently include a sterile aqueous preparation of the active agent, or dispersions of sterile powders of the active agent, which are preferably isotonic with the blood of the recipient. Isotonic agents that can be included in the liquid preparation include sugars, buffers, and sodium chloride. Solutions of the active agent can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

Formulations suitable for oral administration may be presented as discrete units such as tablets, troches, capsules, lozenges, wafers, or cachets, each containing a predetermined amount of the active agent as a powder or granules, as liposomes containing the active agent, or as a solution or suspension in an aqueous liquor or non-aqueous liquid such as a syrup, an elixir, an emulsion, or a draught. The amount of active agent is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations include the active agent dissolved or suspended in one or more media such as mineral oil, DMSO, polyhydroxy alcohols, or other bases used for topical pharmaceutical formulations.

Useful dosages of the active agents can be determined by comparing their in vitro activity and the in vivo activity in animal models, including, for example, the various in vitro and in vivo model systems presented in more detail herein in the examples section. Methods for extrapolation of effective dosages in mice, and other animals, to humans are known in the art.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid and the like; a lubricant such as magnesium stearate; a sweetening agent such as sucrose, fructose, lactose or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

### Kits for Administration of Agents

The present invention also provides a kit for practicing the methods described herein. The kit includes one or more of the agents of the present invention in a suitable packaging material in an amount sufficient for at least one administration. Optionally, other reagents such as buffers and solutions needed to practice the invention are also included. Instructions for use of the packaged agents are also typically included.

As used herein, the phrase “packaging material” refers to one or more physical structures used to house the contents of the kit. The packaging material is constructed by well known methods, preferably to provide a sterile, contaminant-free environment. The packaging material has a label that indicates that the agent(s) can be used for the methods described herein. In addition, the packaging material contains instructions indicating how the materials within the kit are employed to practice the methods. As used herein, the term “package” refers to a solid matrix or material such as glass, plastic, paper, foil, and the like, capable of holding within fixed limits the agent(s). Thus, for example, a package can be a glass vial used to contain appropriate quantities of the agents(s). “Instructions for use” typically include a tangible expression describing the conditions for use of the agent.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Inhibition of MMP-12-Mediated Damage in Alport Syndrome

Herein it is shown that matrix metalloproteinase-12 (MMP-12) is markedly induced in the glomeruli of Alport mice. The degree of induction in glomeruli from Alport mice correlates well with the progression of glomerular disease. The cellular mechanism of MMP-12 induction is identified as monocyte chemoattractant protein-1 (MCP-1)-mediated activation of the CCR2 receptor on glomerular podocytes. Inhibition of MMP-12 with either the inhibitor MMI 270, or the CCR2 receptor antagonist propagermanium, arrests and may reverse the progressive GBM thickening, maintaining the integrity of the glomerular filter. These data suggest that irregular thickening of the GBM in Alport syndrome is caused by proteolytic degradation of the GBM due to elevated expression of MMP-12 in glomerular podocytes. The cellular mechanism of MMP-12 induction (MCP-1 activation of CC chemokine receptor 2 (CCR2) on glomerular podocytes) is novel, quite unexpected, and only previously described in macrophages.

The present example shows that metalloelastase (MMP-12) expression is induced greater than 40-fold in glomerular podocytes of Alport mice, and that suppression of MMP-12 activity decreases renal pathology in the Alport mouse model. Expression of MMP-12 was previously thought to be restricted to macrophages and hypertrophic chondrocytes. Treatment of Alport mice with MMI-270, a inhibitor for MMP-12 resulted in largely restored GBM ultrastructure and function. It was also shown that MMP-12 is induced by MCP-1 activation of the CCR2 receptor on glomerular podocytes of Alport mice, and that inhibition of CCR2 receptor signaling blocks induction of MMP-12 mRNA and prevents GBM damage. Thus, irregular “thickening” of the GBM may represent focal degradation of the GBM resulting from induced MMP-12 activity.

### Mice and Administration of MMP Inhibitors.

The Alport mouse model has been described (Cosgrove et al. (1996) Genes Dev. 10, 2981-2992). The control mice used were normal for both collagen α3(IV) alleles, and are a product of double heterozygote crosses for the Alport mutation. The use of animals in this study was performed in accordance with an approved institutional IACUC protocol. Extreme care was taken to minimize pain and discomfort suffered by the mice. MMP inhibitors were administered between 4 and 7 weeks of age. All drugs were freshly prepared prior to administration. BAY 129566 was emulsified in suspension with 0.5% carboxymethyl cellulose and 4 mg given once a day by oral gavage. MMI-270 was solubilized in 0.9% saline and administered by I.P. injection (50 μg/g body weight) once a day.

### Glomerular Isolation

Isolation of mouse glomeruli was performed as described by Takemoto et al., (Am. J. Pathol. (2002)161, 799-805). The procedure involves cardiac perfusion with deactivated 4.5 μM Dynabeads (Dynal Biotech, Oslo, Sweden), followed by collagenase digestion and glomerular isolation using a magnet. The preparations were found to be consistently >99% pure, allowing reliable assessment of glomerular-specific gene expression in mice.

### Real Time PCR Analysis

Total RNA samples were treated with RNase-free DNase I (Gibco BRL, USA) at 37° C. for 1 hour (hr) in order to remove any contaminating genomic DNA before reverse transcription (RT). Total RNA was reverse-transcribed by using Superscript II (GIBCO BRL) with oligo dt primers. To ensure that the quantitation of MMP transcripts in serial samples was not affected by differences in the amount of RNA added, integrity of RNA, or sample to sample differences in levels of RT-PCR inhibition, an internal control reaction targeting the GAPDH gene was run in multiplex with each reaction, and used to normalize results for MMP transcripts. Primers and TAQman probes for murine GAPDH were purchased from Applied Biosystems (Catologue # 4308313) and used as per the manufacturer's instructions. The data were analyzed using comparative threshold cycle (C_{T}) method. The mRNA quantity for the control is expressed as 1× sample and all other quantities from Alport samples are expressed as fold difference relative to the controls. No measurable fluorescence signal was detected in repeated RT-PCR runs in which the reverse transcriptase was omitted from the reaction mixture. Primers were tested by standard endpoint RT-PCR, and the single band obtained was sequence verified. Real time RT-PCR was performed on a TaqMan ABI 7000 Sequence Detection System (Applied Biosystems, Foster City, Calif.).

PCR was carried out with TaqMan Universal PCR Master Mix (Applied Biosystems), which contained AmpliTaq Gold DNA polymerase, AmpErase urasil-N-glycosylate, dNTPs with dUTP, passive Reference, and optimized buffer components. AmpErase urasil-N-glycosylate treatment prevented the possible reamplification of carryover PCR products. Each target molecule was coamplified with primers and TaqMan probe for GAPDH in the same PCR tube. The total volume of the PCR reaction was 50 microliters (μl). The final concentration of each oligonucleotide in the PCR reaction was as follows: GAPDH primers, 100 nanomolar (nM); primers for target molecules, 900 nM; TaqMan probe for GAPDH, 200 nM; and TaqMan probe for the target molecules, 250 nM.

Sequences and fluorescent dye of PCR primers and
TaqMan probes:
MMP-2:

(SEQ ID. NO:1)

| | |
|---|---|
| Sense | 5′-GTT TAT TTG CCC GAC AGT GAC A-3′ |

(SEQ ID. NO:2)

| | |
|---|---|
| Antisense | 5′- AGA ATG TGG CCA CCA GCA A-3′ |

(SEQ ID. NO:3)

| | |
|---|---|
| Probe | 5′-6FAM-CCA CGT GAC AAG CC-MGBNFQ-3′ |
| MMP-9: | |

(SEQ ID. NO:4)

| | |
|---|---|
| Sense | 5′-CCA AGG GTA CAG CCT GTT CCT-3′ |

(SEQ ID. NO:5)

| | |
|---|---|
| Antisense | 5′-GCA CGC TGG AAT GAT CTA AGC-3′ |

(SEQ ID. NO:6)

| | |
|---|---|
| Probe | 5′-6FAM-ACT CGT GCG CTG CC-MGBNFQ-3′ |
| MMP-12: | |

(SEQ ID. NO:7)

| | |
|---|---|
| Sense | 5′-GCC ACA CTA TCC CAG GAG CAT ATA-3′ |

(SEQ ID. NO:8)

| | |
|---|---|
| Antisense | 5′-AGC TGC ATC AAC CTT CTT CAC A-3′ |

(SEQ ID. NO:9)

| | |
|---|---|
| Probe | 5′-6FAM-ATG CAG AGA AGC CC-3′ MGBNFQ-3′ |
| MMP-14: | |

(SEQ ID. NO:10)

| | |
|---|---|
| Sense | 5′-GAG GAG AGA TGT TTG TCT TCA AGG A-3′ |

(SEQ ID. NO:11)

| | |
|---|---|
| Antisense | 5′-GGG TAT CCA TCC ATC ACT TGG TTA-3′ |

(SEQ ID. NO:12)

| | |
|---|---|
| Probe | 5′-6FAM-TCC TCA CCC GCC AGA G-MGBNFQ-3′ |

TaqMan Rodent GAPDH Control Reagents (Cat # 4308313) containing the primers and VIC-probe was purchased from Applied Biosystems.

Thermal cycling was initiated with incubation at 50° C. for 2 minutes (min) and 95° C. for 10 min for optimal EmpErase UNG activity and activation of AmpliTaq Gold DNA polymerase, respectively. After this initial step, 40 cycles were performed. Each PCR cycle consisted of heating at 95° C. for 15 seconds (sec) for melting and 60° C. for 60 sec for annealing and extension. All controls consisting of double distilled (dd) H₂O were negative for target and housekeeping genes.

### Conventional PCR

CCR2 Primers: Annealed at 58° C., 35 cycles 199 bp

(SEQ ID. NO:13)

Forward: 5′-CAC GAA GTA TCC AAG AGC TT-3′

(SEQ ID. NO:14)

Reverse: 5′-CAT GCT CTT CAG CTT TTT AC-3′
MCP-1: Annealed at 60° C. for 30 cycles 519 bp

(SEQ ID. NO:15)

Forward: 5′-AGA GAG CCA GAC GGA GGA AG-3′

(SEQ ID. NO:16)

Reverse: 5′-GTC ACA CTG GTC ACT CCT AC-3′

Data are expressed as mean ±SD. Differences between means were tested for significance using Student's t-test. Differences were considered significant at the level of P<0.05.

### Immunohistochemistry

Cryosections (4 micromolar (μM)) of kidneys from 7-week-old normal and Alport mice were air dried, fixed by immersion in ice cold acetone, and subjected to immunohistochemical staining analysis using antibodies specific for MMP-3 (rabbit polyclonal anti-human MMP-3, a gift from Dr. Z. Gunza-Smith, Miami, Fla., used at 1:200 dilution), MMP-12 (rabbit polyclonal antibody against mouse MMP-12, kindly provided by Yoshikatsu Kaneko, used at 1:100 dilution), type IV collagen α1/2 chains (rabbit polyclonal against mouse type IV collagen, Biodesign, Inc., Saco, Me., used at 1:200 dilution), fibronectin (rabbit polyclonal against human plasma fibronectin, Sigma Chemical Co., St. Louis, Mo., used at 1:200). Anti-CD31 antibodies were directly conjugated to Alexa 568 (Molecular Probes, Eugene Oreg.) and purchased from Immunotech (Marseille, France). For dual immunofluorescence immunostaining, this antibody was added to the mixture containing the secondary antibody. All antibodies were diluted into 7% non-fat dry milk in PBS to reduce non-specific binding. Primary antibodies were allowed to react for two hours at room temperature in a humidified chamber. After three five-minute washes in PBS, slides were incubated with FITC-conjugated secondary antibodies for 1 hour at room temperature (goat anti-rabbit, Vector Laboratories, Burlingame, Mass., used at 1:200). The sections were cover-slipped, sealed, and imaged. Images were collected using a Cytovision Ultra Image analysis system interfaced with an Olympus BH-2 fluorescence microscope.

### Northern Blot Analysis

Northern blots analysis was performed as described previously (Cosgrove et al., 1996, Genes Dev. 10, 2981-2992). Ten micrograms of total glomerular RNA was fractionated on 1% agarose formaldehyde gels and transferred to nylon membranes. Probes were either a gel purified PCR fragment of the MMP-12 transcript (see the primers and probes described herein), or the DECA template for mouse β-actin (Ambion, Inc., Austin Tex.). Probes were labeled with ³²P-dCTP using either random primers or the DECA method provided by the manufacturer. Hybridizations were carried out overnight at 50° C. using ULTRAhyb hybridization buffer (AMBION), and the membranes washed according to the manufacturers instructions. Membranes were exposed to X-ray film overnight.

### In Situ Hybridization

Riboprobe Preparation: A 631 bp fragment of the mouse MMP-12 cDNA was amplified from reverse transcribed 13 day mouse embryonic RNA using the primers listed above for real-time PCR. The resulting fragment was cloned into the pCRII topo cloning vector (Invitrogen) and sequence verified. Fifteen micrograms of this plasmid was linearized using HindIII to provide a 5′ overhang. DNA was isolated using phenol/chloroform extractions. One microgram (1 μg) of DNA was labeled as recommended in the Boehringer Mannheim DIG Labeling Kit using T7 polymerase. Spotting the probe and labeled control onto nylon membrane and developing as recommended in the nonradioactive in situ hybridization application manual (Roche) estimated the labeling yield. For hybridization, 6 micrometer (μm) paraffin sections were digested in 3 μg/ml Proteinase K in 0.1 molar (M) Tris pH 7.5 for 10 min at 37° C. They were prehybridized for 1 hour at 45° C. in: 50% deionized formamide, 2×SSC, 10 Tween 20, and 1 milligram (mg) E. coli tRNA. The hybridization solution consisted of: 50 nanogram (ng) heat-denatured ribroprobe, 50% deionized form amide, 8% dextran sulfate, 10% Tween-20, 2×SSC, 20% tRNA, and 10 mg/ml boiled salmon sperm. Slides were hybridized at 45° C. overnight. The DIG Wash and Block Buffer Set was used to develop the slides in conjunction with the color substrate solution to which we added 25 millimolar (mM) Levamisole.

### Electron Microscopy

Transmission electron microscopy was performed as previously described (Cosgrove et al., 1996, Genes Dev. 10, 2981-2992). Ultrastructural localization studies of type IV collagen were performed essentially as previously described (Bhattacharya et al., 2002). Kidneys from 7-week-old control and Alport mice were fixed by heart perfusion with 2% paraformaldehyde in PBS, and post-fixed in this same solution overnight. Ultrathin (70 nm) sections were reacted for 4 hours at room temperature with goat anti-collagen IV antibodies (Southern Biotechnology, Birmingham, Ala.). After six 10 minute washes in PBS, specimen were reacted for 2 hours at room temperature with 10 nanometers (nm) gold-conjugated anti-rabbit antibodies (Vector Laboratories, Burlingame, Calif.). Grids were washed as before, air-dried, counterstained with uranyl acetate, and examined using transmission electron microscopy.

### Western Blot Analysis.

Isolated glomeruli were lysed in RIPA (Radio Immuno Precipitation Assay) lysis buffer, consisting of 0.1% SDS, 0.5% deoxycholate, 1% Nonidet P-40, 100 mM NaCl, and 10 mM Tris.Cl, pH 7.4 immunoprecipitation buffer containing protease inhibitors (P8340, Sigma Chemical Co., St. Louis, Mo.). The lysate was transferred to a microcentrifuge tube and centrifuged at 15,000 g for 10 minutes at 4° C. The supernatant was assayed for total protein using a commercial Bradford microplate assay (Pierce Biochemicals, Rockford, 1H). Antisera (goat anti-human CCR2, Santa Cruz Biotechnology, Inc, Santa Cruz, Calif.) was added to 10 grams of protein and incubated overnight at 4° C. Protein A-Sepharose CL-4B beads (I 5 microliter per milliliter (μl/ml) of a 50% slurry) were added, incubated on a rocking platform for 1 hr at 4° C., pelleted by centrifugation and washed six times with 10 mM NaCl and once with 100 mM NaCl. The Protein A-Sepharose 4B Cl was resuspended in gel loading buffer, boiled and centrifuged. Immunoprecipitated protein extract for each sample was electrophoresed into 12% SDS-polyacrylamide gels (SDS-PAGE) and transferred to 0.45 μm PVDF immobilon P transfer membranes (Sigma, St. Louis, Mo.). Membranes were quenched at 4° C. overnight in a solution of TBST (Tris-buffered saline+0.5% Tween 20; Fisher Scientific, Pittsburgh, Pa.) and 5% BSA (bovine serum albumin; Sigma, St Louis, Mo.) for blocking nonspecific binding. Anti-CCR2 antibody was diluted 1:1000 in a solution of TBST and 3% BSA and the blots were incubated in this solution overnight. After several washes in a solution of TBST, the blots were incubated with a solution of TBST containing an anti-goat secondary antibody (horse-radish peroxide conjugated; Sigma, St. Louis, Mo.), diluted 1:20,000 for 1 hour at room temperature. The blots were then washed several times in TBST, reacted with an ECL (Enhanced Chemiluminescence kit; Amersham Biosciences Corp, Piscataway, N.J.) and exposed to X-ray films.

Treatment of Mice with the CCR2 Antagonist, Propagermanium

Propagermanium (3-oxygemylpropinic acid polymer, Sanwa Kagaku Kenkyusho Co., Nagoya, Japan) was administered orally (10 milligrams per kilogram (mg/kg) in 1% gelatin) by gavage once daily starting at 5 weeks of age, and kidneys harvested at 7 weeks of age. Three animals per group, Alport mice and control litter mates, gavaged with drug or vehicle only, were analyzed.

### Results

MMP-12 Expression is Markedly Induced in Glomeruli from Alport Mice and Humans

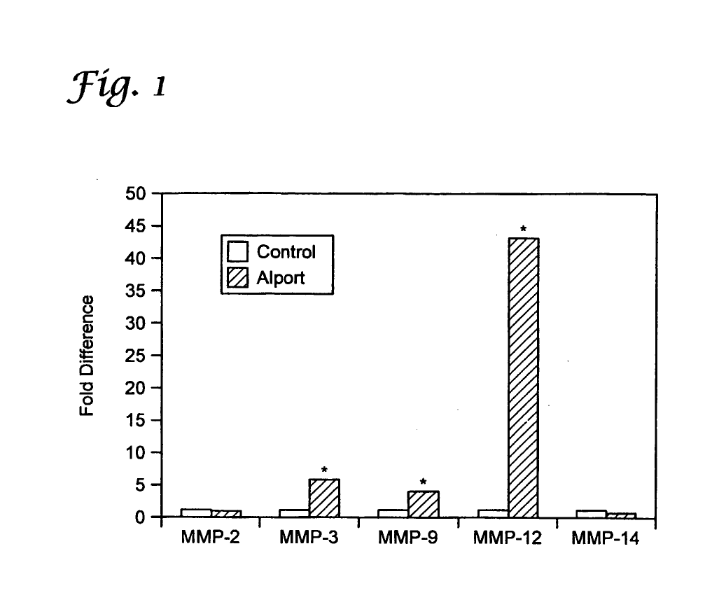

A newly described glomerular isolation technique (Takemoto et. al. (2002) Am. J. Pathol. 161, 799-805) was employed to obtain pure glomerular RNA preparations from normal mice and Alport mice at 7 weeks of age (7 week old Alport mice have advanced glomerular disease). Total RNA was prepared and analyzed using real-time RT-PCR for expression of the MMPs as shown in FIG. 1, using an internal quenched FAM conjugated primer method. GAPDH was run in multiplex in all reactions as an internal control for RNA loading. Three independent glomerular preparations were analyzed in triplicate. The results provided in FIG. 1 show that mRNAs encoding MMP-2 and MMP-14 were not significantly changed in diseased Alport glomeruli relative to control glomeruli. In contrast, the mRNAs encoding MMP-3 and MMP-9 were 4- to 5-fold higher in Alport glomeruli relative to controls. Remarkably, expression of MMP-12 mRNA was greater than 40-fold higher in Alport mice compared to normal mice. MMP-7 was also analyzed; however, no expression of MMP-7 was observed in glomeruli (data not shown).

While induction of MMP-3 and MMP-9 are well documented in glomerular disease (Suzuki et. al. (1997) Kidney Int. 52, 111-119 and Urushihara et al. (2002) Nephrol Dial Transplant. 17, 1189-1196), expression of MMP-12 in normal or diseased glomeruli has not been documented, with the exception of autoimmune glomerulonephritis, where the MMP-12 expression was due to infiltrating macrophages (Kaneko et. al. (2003) J. Immunol. 170, 3377-3385). Kidneys from normal and 7-week-old Alport mice were immunostained using antibodies specific for either MMP-3 or MMP-12.

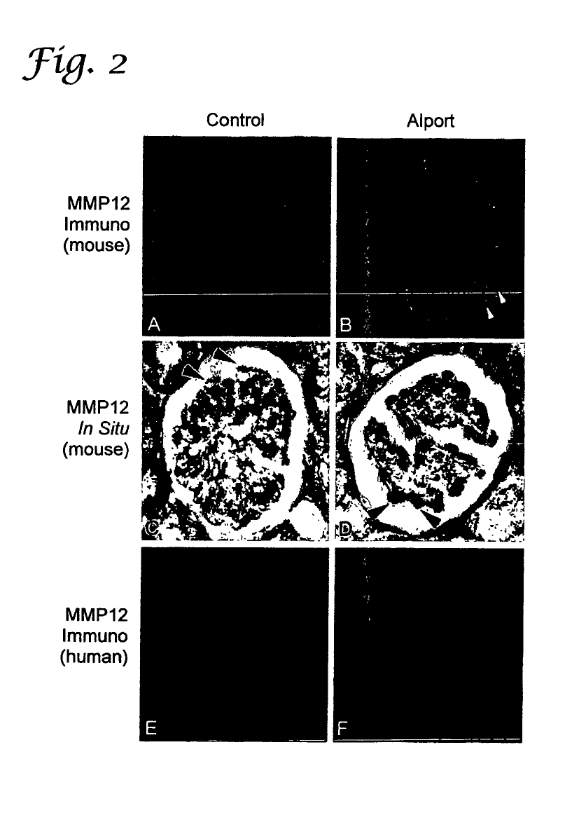

The results in FIG. 2 (A and B) show that MMP-12 was induced in Alport mice relative to controls. Arrows in panel B denote the strongest immunostaining for MMP-12 was observed around what appear to be glomerular podocytes. To confirm that induction of MMP-12 was indeed occurring in the glomerular podocytes, in situ hybridization analysis was performed. The results in FIG. 2 panel D show that a strong signal was observed for MMP-12 mRNA in cells lining the outer circumference of the glomerulus (arrows), consistent with localization within the glomerular podocytes. MMP-12 mRNA was not observed in the podocyte of normal littermates (FIG. 2, Panel C, arrows). FIG. 2 (panel F) shows that human Alport glomeruli express high levels of MMP-12, while MMP-12 immunostaining is not observed in glomeruli from normal humans (FIG. 2 panel E).

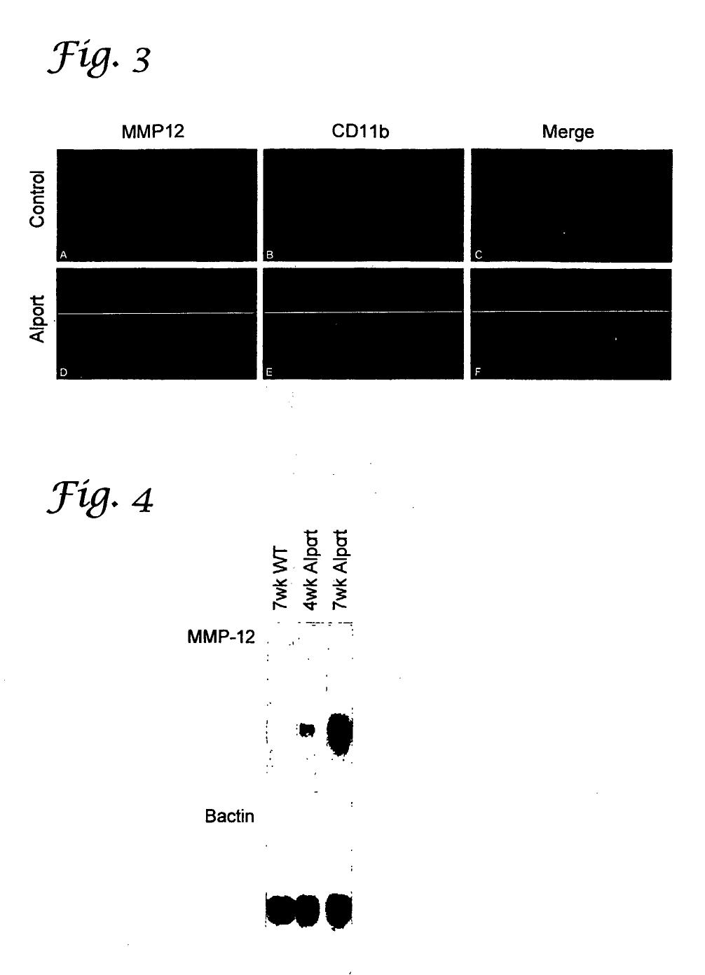

It was possible that the observed expression of MMP-12 in Alport glomeruli might represent expression by infiltrating macrophages. To address this, dual immunofluorescence analysis was performed using antibodies specific for MMP-12 and CD11b (a specific marker for monocytes and macrophages). The results provided in FIG. 3 show that there were no monocytes or macrophages present in Alport glomeruli, and that the interstitial macrophages (CD11b positive cells in red) do not express MMP-12.

To determine whether MMP-12 mRNA is inducible as a function of glomerular disease progression, glomerular RNA was analyzed by Northern blot. Glomeruli from three independent preparations were combined and total RNA was fractionated on MOPS gels, transferred to nylon membranes and hybridized to a radiolabeled probe specific for MMP-12. The results in FIG. 4 show that MMP-12 mRNA is inducible, as evidenced by the absence of expression of RNA from control mice and the obvious presence of signal in 4-week-old Alport mice. The signal was markedly intensified by 7 weeks, indicating a progressive induction of MMP-12 RNA during the course of glomerular disease progression.

### Inhibition of MMP-12 Activity Restores Normal GBM Architecture and Glomerular Function in Alport Mice.

MMP-12 has broad substrate specificity that includes many of the known basement membrane proteins, including type IV collagen, laminins, entactin, and proteoglycans (Gronski et al. (1997) J. Biol. Chem. 272, 12189-12194). Thus it appeared likely that such a significant increase in MMP-12 might influence the functional integrity of the GBM in Alport glomeruli. To test this, two different inhibitors for the MMPs were used. As noted in Table 1, BAY 129566 inhibits MMP-2,3,9, and 14, but not MMP-12 (Gatto et. al., (1999) Clin. Cancer Res. 5, 3603-3607 and Hidalgo et al. (2001) J. of the Nat. Cancer Ins. 93, 178-193). MMI-270 inhibits MMP-2, MMP-3, MMP-9, MMP-14, and MMP-12 (MacPherson et al. (1997) J. Med. Chem. 40, 2525-2532). This activity underlies the clinical application of the compound, which is primarily aimed at treating lung fibrosis where macrophage-derived MMP-12 has been shown to underlie fibrogenesis.

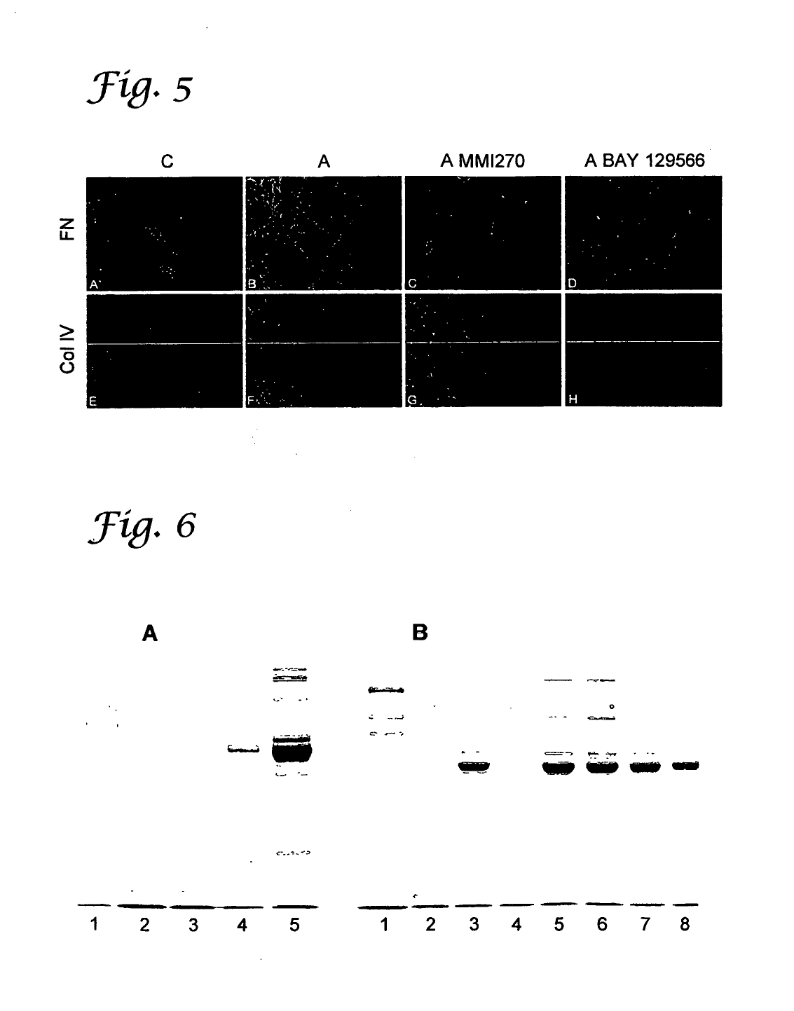

To assess the effect of these two compounds on Alport renal disease progression, Alport mice were administered either MMI 270 or BAY 129566 from 4 to 7 weeks of age. The animals were transcardially perfused with PBS and the kidneys harvested. Cryosections were analyzed by immunofluorescence microscopy to assess for the degree of glomerular and tubulointerstitial damage using antibodies specific for collagen IV α1 and α2 chains, and fibronectin. The results provided in FIG. 5 illustrate that that the MMI 270-treated animals appeared to have very little glomerular or interstitial disease compared to the age-matched untreated Alport mice (compare panels C and G with B and F). This observation is in contrast to the BAY 129566-treated animals, which showed the same degree of renal pathology as untreated Alport mice (compare panels D and H with B and F). This observation was further substantiated by the dramatic reduction of proteinuria in MMI-270-treated Alport mice (FIG. 6), suggesting that this inhibitor largely preserved the integrity of the glomerular filter. If given at a later stage of glomerular disease development (6 to 7 weeks of age), MMI-270 arrests the progressive increase in proteinuria normally observed (FIG. 6 panel B), suggesting MMP-12 inhibition will arrest further progression of glomerular pathogenesis even in an advanced disease state.

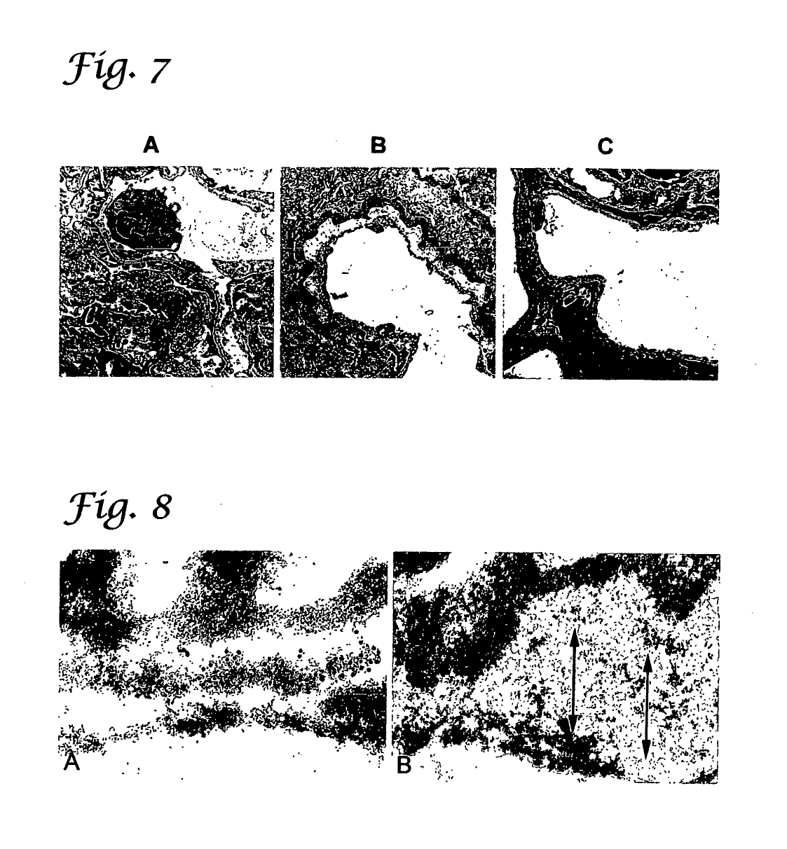

Electron microscopic analysis of the glomerular basement membranes in MMI 270-treated mice revealed near complete restoration of normal glomerular basement membrane architecture in most of the glomeruli examined. FIG. 7 shows typical observed improvement of the GBM in a glomerular capillary loop. Normal GBM is shown in panel A. Untreated Alport mice showed marked irregular thickening of the GBM (FIG. 7B). MMI-270 treated mice showed a remarkable restoration of normal glomerular basement membrane architecture (FIG. 7C). This observation is important. Restoration of the glomerular basement membrane architecture likely underlies restoration of glomerular function as measured by proteinuria. Proteolytic degradation of the GBM in Alport syndrome might underlie the progressive irregular GBM damage and podocyte foot process effacement. Type IV collagen from Alport kidneys is more susceptible to proteolytic degradation in vitro than type IV collagen from healthy kidneys (Kalluri et al. (1997) J. Clin. Invest. 99, 2470-2478, presumably owing to the reduced number of interchain disulfide crosslinks (Gunwar et al. (1998) J. Biol. Chem. 273, 8767-8775).

If elevated expression of MMP-12 leads to progressive proteolytic destruction of the GBM, the observed GBM irregularities might represent areas of focal degradation. In an attempt to visualize this directly, colloidal gold immunocytochemistry was employed using antibodies against type IV collagen α1 and α2 chains. Colloidal gold ultrastructural localization was employed to examine the integrity of the type IV collagen network in Alport GBM. Antibodies were against type IV collagen a1 and a2 chains. Secondary antibodies were conjugated to 10 nm colloidal gold beads. The results provided in FIG. 8 represent two different regions of the GBM in an affected Alport glomerular capillary. Panel A represents a region with normal ultrastructure. Here the immunogold labeling was localized along the lamina densa of the GBM. Panel B illustrates a region of focal thickening in the GBM. In contrast to panel A, here the immunogold shows an irregular localization pattern, with labeling clustering either along the epithelial or endothelial boundaries of the GBM. The arrows in panel B denote a consistent observation. When immunogold clusters are observed on the epithelial boundary, they are relatively absent in the opposing endothelial boundary and vice versa. This is consistent with splitting and cleavage of the basement membrane collagen superstructure, which would be expected upon endoproteolytic damage.

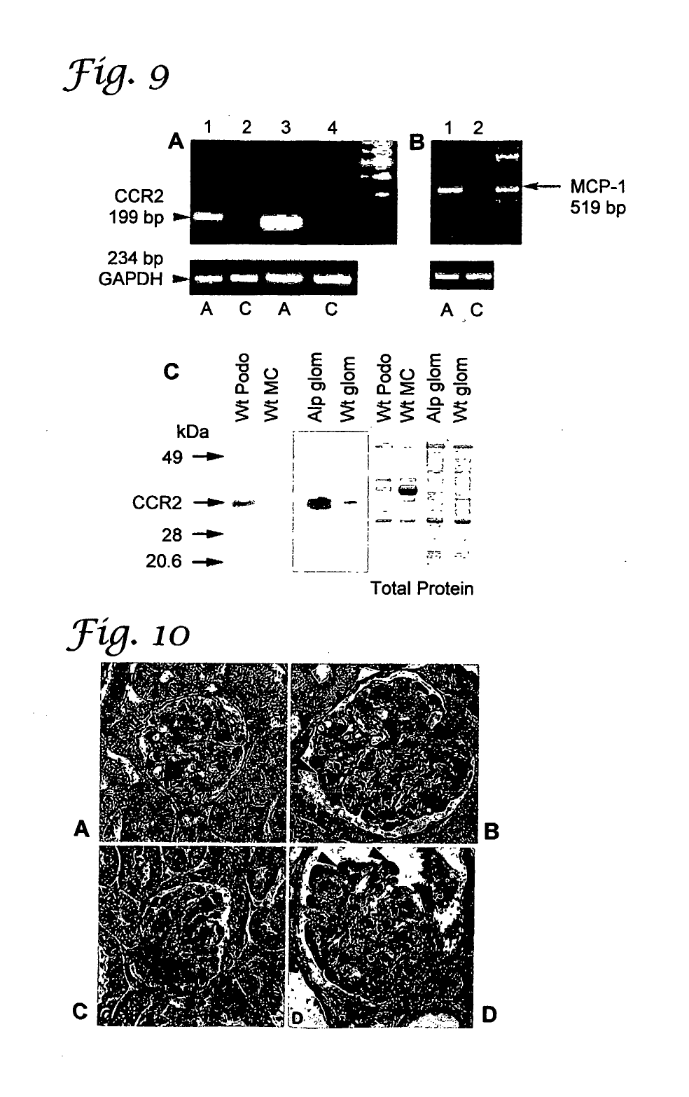

The cellular mechanism of MMP-12 induction in Alport glomeruli is MCP-1-mediated activation of the CCR2 receptor on glomerular podocytes. Blocking the CCR2 receptor reduces MMP-12 expression and restores the GBM architecture in Alport mice. The cellular mechanism of MMP-12 induction in macrophages has been linked to granulocyte/monocyte chemoattractive factor (GM-CSF), interleukin-1beta (IL-β), and monocyte chemoattractive protein-1 (MCP-1) (Wu et al. (2003) Genes Cells 8, 225-234). Glomeruli from normal mice and Alport mice were examined for expression of these regulatory systems. The results in FIG. 9 (Panel A) show that CCR2 mRNA was markedly up-regulated in glomeruli from Alport mice relative to normal mice (lanes 3 and 4). Cultured glomerular podocytes from Alport mice also showed elevated expression of CCR2 mRNA relative to podocytes from normal littermates (lanes 1 and 2). Western blot analysis confirmed that CCR2 protein was elevated in Alport glomeruli relative to normal glomeruli (FIG. 9, Panel C). In addition, CCR2 protein was observed in extracts of cultured podocytes, but was notably absent from cultured mesangial cells (FIG. 9, Panel C). MCP-1 (also called CCL2), the chemokine ligand for CCR2, was also induced in glomeruli from Alport mice relative to glomeruli from normal mice (FIG. 9, Panel B). Thus a chemokine/ligand system is present and induced in Alport glomeruli, which may constitute the cellular mechanism of MMP-12 induction in Alport glomerular podocytes.

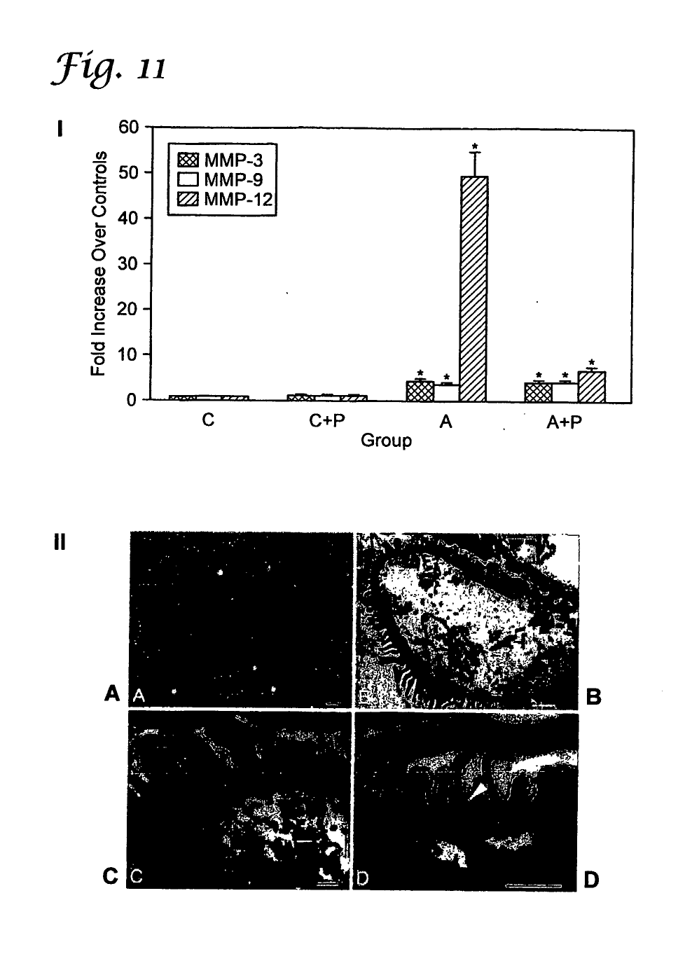

To test whether this mechanism is indeed active in Alport glomeruli, a specific inhibitor of CCR2, propagermanium (3-oxygermylpropionic acid), was employed. This compound inhibits CCR2 receptor by targeting glycosylphosphatidlinositol-anchored proteins that are closely associated with CCR2 (Yokochi et al. (2001) J. Interferon Cytokine Res. 21, 389-398). The drug was given by oral gavage to three Alport mice and three control mice starting at 5 weeks of age and the glomeruli harvested from the kidneys at 7 weeks of age. As controls, both Alport mice and wild type mice were gavaged with vehicle only. RNA was isolated from glomeruli and analyzed in triplicate for expression of the MMPs using real time PCR. The results in FIG. 10 (Panel 1) show that MMP-3, MMP-9, and MMP-12 were all induced in glomeruli from Alport mice gavaged with vehicle relative to normal controls, which is both qualitatively and quantitatively consistent with the results in FIG. 1. In the propagermanium treated Alport mice, induction of both MMP-3 and MMP-9 mRNAs was unaffected by the drug, whereas MMP-12 mRNA induction was reduced from 50-fold in glomeruli from vehicle-treated mice, to 6-fold in glomeruli from propagermanium-treated Alport mice. Renal cortex from these same mice were examined by transmission electron microscopy. FIG. 10 (Panel II) shows that the reduced expression of MMP-12 was sufficient to restore normal GBM architecture. This restoration results in reestablishment of the slit diaphragm (FIG. 10, Panel D, arrow) and the reappearance of healthy fenestrated endothelium in the glomerular capillary tuft. These data establish MCP-1 activation of CCR2 on glomerular podocytes as the cellular mechanism underlying MMP-12 activation in Alport glomeruli, and illustrate that it is indeed MMP-12, and not MMP-3 or MMP-9, that is responsible for glomerular basement membrane destruction in Alport syndrome.

### Discussion

Example 1 provides two distinct lines of evidence that support the notion that elevated MMP-12 causes GBM destruction. First, comparative studies provided herein, using two different inhibitors of matrix metalloproteinases, show that MMI 270 prevents or reverses GBM damage, while BAY-12-9566 has no effect. While these two compounds share inhibitory activity for a number of MMPs, MMI-270 inhibits MMP-12, while BAY-12-9566 does not (Table I). In addition, a second body of evidence is provided that describes an unexpected cellular mechanism underlying MMP-12 activation in glomerular podocytes; namely, monocyte chemoattractant protein-1 (MCP-1) activation of CCR2 on glomerular podocytes. It can be seen that MMP-12 induction is very significant in the GBM pathogenesis of Alport syndrome, and that irregular “thickening” of the GBM, and the associated loss of glomerular filter integrity, results primarily from proteolytic degradation of the GBM by MMP-12.

TABLE 1
profile of MMP inhibitory effects for the drugs used in this study.

| DRUG | MMP-2 | MMP-3 | MMP-9 | MMP-12 | MMP-14 |
|---|---|---|---|---|---|
| BAY 12-9566 | + | + | + | − | − |
| MMI 270 | + | + | + | + | + |

It is notable that both MMP-3 and MMP-9 expression levels are about 5-fold elevated in 7-week-old Alport mice relative to controls. FIG. 5 shows that administration of BAY 129566 had no obvious effect on the progression of Alport renal disease, suggesting that elevated expression of MMP-3 and MMP-9 does not play an important role. This is in contrast to related studies where elevated MMP-9 over-expression was shown to be protective in anti-glomerular basement membrane nephritis (Lelongt et al. (2001) J. Exp. Med. 193, 793-802), but consistent with the observation that an MMP-9 null background does not influence the progression of Alport renal disease in the mouse model (Andrews et al. (2002) Am. J. Pathol. 160, 721-730). Both MMP-3 and MMP-9 knockout mice are viable, and have no known functional deficit in the kidney (Rudolph-Owen et al. (1997) Endocrinology 138, 4902-4911).

The role of MMP-12 in Alport glomerular pathogenesis is quite unexpected. Previous studies suggest expression of MMP-12 is highly restricted, having only been described in macrophages (Vos et al. (2003) J. Neuroimmunol. 138, 106-114 and Kaneko et. al. (2003) J. Immunol. 170, 3377-3385), hypertrophic osteoblasts (Hou et. al. (2004) Bone 34, 37-47), and vascular smooth muscle cells (Wu et al. (2003) Genes Cells 8, 225-234). Expression of MMP-12 by a differentiated epithelial cell has not been previously demonstrated. Elevated glomerular expression of MMP-12 has been shown in autoimmune glomerulonephritis, however the source of MMP-12 in this study was shown to be infiltrating macrophages (Kaneko et. al. (2003) J. Immunol. 170, 3377-3385). Nonetheless, Kaneko et al. supports the notion that overexpression of MMP-12 in glomeruli can lead to pathology. However, the results illustrated by FIG. 3 show that there are no macrophages in Alport glomeruli, and that the interstitial monocytes present in Alport kidneys are immuno-negative for MMP-12. Immunofluorescence data shown in FIG. 2D indicate the source of MMP-12 expression in Alport glomeruli may be glomerular podocytes; however, mesangial cells might also be involved.

The observed 40-fold induction of MMP-12 in Alport glomeruli, and the arrested glomerular pathogenesis in animals treated with MMP-12 inhibitor MMI-270, supports a role for proteolytic degradation underlying the irregular rarification of the GBM. It has been previously shown that the GBM from patients with Alport syndrome is more susceptible to endoproteolysis (Kalluri et al. (1997) J. Clin. Invest. 99, 2470-2478). Biochemical analysis shows that type IV collagen networks comprised of the a 3(IV), a 4(IV) and a 5(IV) chains are more heavily crosslinked than those comprised solely of collagen α1 (IV) and α2(IV) chains (Gunwar et al. (1998) J. Biol. Chem. 273, 8767-8775). This could account for the enhanced resistance of normal GBM to proteolysis. Thus, elevated MMP-12 combined enhanced susceptibility to endoproteolysis due to its collagen α1(IV) and α2(IV) composition may both contribute to the observed ultrastructural dysmorphology of the GBM in Alport syndrome.

As noted earlier, type IV collagen in the extracellular matrix from Alport kidneys may be more susceptible to endoproteolytic cleavage than that from normal kidneys (Kalluri et al. (1997) J. Clin. Invest., 99, 2470-2478). The results described herein further demonstrate that MMP-12 is overexpressed in the glomeruli of an Alport mouse model. While not intending to be bound by theory, overexpression of metalloproteinase and/or increased vulnerability of the ECM to proteinase degradation may be involved in the formation of irregular thickness of the glomerular basement membrane associated with Alport syndrome. Accordingly, decreasing the level of matrix metalloproteinase-12 activity may decrease degradation of the ECM in the GBM in a subject with Alport syndrome.

### Example 2

### Metalloelastase (MMP-12) Induction in Podocytes in Alport Glomerular Pathogenesis

Glomerular pathogenesis in Alport syndrome is characterized by irregular thickening, thinning and splitting of the glomerular basement membrane and podocyte foot process effacement. Ultrastructural damage of the GBM may be due to proteolytic degradation, presumably owing to decreased cross-linking of the GBM collagen. Using magnetic bead isolation of glomeruli combined with real time PCR, it was found that a number of MMPs are induced in Alport glomeruli. Most notable was MMP-12, where mRNA was more than 40-fold induced by both real time PCR and northern blot analysis. Immunofluorescence analysis suggests that induction of MMP-12 occurs primarily in the podocytes of Alport mice. Two different inhibitors were employed to explore the role of MMPs in glomerular pathogenesis in the collagen α3(IV)-null mouse model. Treatment of Alport mice with BAY 129566 (Bayer Corporation) from 4 to 7 weeks of age did not significantly affect the course of glomerular disease progression, while treatment with NMI 270 (Novartis Corporation) showed a profound influence. These two inhibitors, both with wide MMP substrate specificity, differ primarily in that NMI 270 inhibits MMP-12, while BAY 129566 does not. NMI 270 treated mice had drastically reduced levels of proteinuria, markedly improved GBM ultrastructure, and significantly reduced interstitial disease when compared to Alport controls or BAY 129566 treated Alport mice. When NMI 270 was administered to Alport mice from 6 to 7 weeks of age, the increase in proteinuria normally observed was arrested, suggesting immediate benefits of drug treatment even in animals with more advanced disease. Combined, these data suggest that elevated MMP-12 levels are important for the mechanism of Alport glomerular pathogenesis, and support the hypothesis that increased proteolysis of the GBM underlies the observed ultrastructural and functional changes associated with progressive Alport glomerular pathogenesis.

The complete disclosure of all patents, patent applications, and publications, and electronically available material (including, for instance, nucleotide sequence submissions in, e.g., GENBANK and REFSEQ, and amino acid sequence submissions in, e.g., SWISSPROT, PIR, PRF, PDB, and translations from annotated coding regions in GENBANK and REFSEQ) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### Sequence Listing Free Text

SEQ ID. NO:1; MMP-2 sense primer polynucleotide
SEQ ID. NO:2; MMP-2 antisense primer polynucleotide
SEQ ID. NO:3; MMP-2 probe polynucleotide
SEQ ID. NO:4; MMP-9 sense primer polynucleotide
SEQ ID. NO:5; MMP-9 antisense primer polynucleotide
SEQ ID. NO:6; MMP-9 probe polynucleotide
SEQ ID. NO:7; MMP-12 sense primer polynucleotide
SEQ ID. NO:8; MMP-12 antisense primer polynucleotide
SEQ ID. NO:9; MMP-12 probe polynucleotide
SEQ ID. NO: 10; MMO-14 sense primer polynucleotide
SEQ ID. NO:11; MMP-14 antisense primer polynucleotide
SEQ ID. NO:12; MMP-14 probe polynucleotide
SEQ ID. NO:13; CCR2 forward primer polynucleotide
SEQ ID. NO:14; CCR2 reverse primer polynucleotide
SEQ ID. NO:15; MCP-1 forward primer polynucleotide.
SEQ ID. NO:16; MCP-1 reverse primer polynucleotide
SEQ ID. NO:17; matrix metalloproteinase-12 polypeptide
SEQ ID. NO:18; matrix metalloproteinase-12 substrate polypeptide
SEQ ID. NO:19; CCR2 receptor polypeptide
SEQ ID. NO:20; MCP-1 chemokine polypeptide

1-40. (canceled)

41. A method for treating glomerular basement membrane disease in a subject, comprising administering a matrix metalloproteinase-12 (MMP-12) inhibitor, a CCR2 receptor inhibitor, a MCP-1 inhibitor, or a combination thereof to the subject.

42. The method of claim 41, wherein the glomerular basement membrane disease is Alport syndrome.

43. The method of claim 42, wherein the inhibitor decreases the irregularity of the width of the glomerular basement membrane associated with Alport syndrome.

44. The method of claim 41, wherein the inhibitor decreases the degradation of extracellular matrix in the glomerular basement membrane.

45. The method of claim 41, wherein administering the inhibitor decreases matrix metalloproteinase-12 activity in glomerular podocytes.

46. The method of claim 41, wherein the inhibitor is a non-peptidic inhibitor.

47. The method of claim 46, wherein the inhibitor is a matrix metalloproteinase-12 inhibitor and is an arylsulfonamide substituted hydroxamic acid derivative.

48. The method of claim 47, wherein the arylsulfonamide-substituted hydroxamic acid is MMI-270.

49. The method of claim 46, wherein the inhibitor is a matrix metalloproteinase-12 inhibitor and is selected from the group consisting of thiophene amino acid derivatives, fluorothiophene derivatives, and 1-carboxymethyl-2-oxo-azepan derivatives.

50. The method of claim 41, wherein the inhibitor is an antibody.

51. The method of claim 41, wherein the inhibitor is an oligonucleotide.

52. The method of claim 41, wherein the inhibitor is a CCR2 receptor inhibitor and is an organogermanium compound.

53. The method of claim 52, wherein the organogermanium compound is 3-oxygemylpropinic acid polymer.

54. The method of claim 41, wherein the inhibitor is administered orally, intravenously, intramuscularly, intraperitoneally, and/or subcutaneously.

55. The method of claim 41 further comprising administering one or more additional treatment modalities.

56. The method of claim 55 wherein the additional treatment modality comprises kidney dialysis.

57. The method of claim 55 wherein the additional treatment modality comprises the administration of a corticosteroid.

58. The method of claim 55 wherein the additional treatment modality comprises the administration of a non-steroidal anti-inflammatory drug (NSAID).


SEQUENCE LISTING (20 sequences)

SEQ ID NO: 1 - DNA, 22 bp, artificial sequence
  chemically synthesized  primer
gtttatttggcggacagtgaca

SEQ ID NO: 2 - DNA, 19 bp, artificial sequence
  chemically synthesized primer
agaatgtggccaccagcaa

SEQ ID NO: 3 - DNA, 14 bp, artificial sequence
  chemically synthesized probe
ccacgtgacaagcc

SEQ ID NO: 4 - DNA, 21 bp, artificial sequence
  chemically synthesized primer
ccaagggtacagcctgttcct

SEQ ID NO: 5 - DNA, 21 bp, artificial sequence
  chemically synthesized primer
gcacgctggaatgatctaagc

SEQ ID NO: 6 - DNA, 14 bp, artificial sequence
  chemically synthesized probe
actcgtgcgctgcc

SEQ ID NO: 7 - DNA, 24 bp, artificial sequence
  chemically synthesized primer
gccacactatcccaggagcatata

SEQ ID NO: 8 - DNA, 22 bp, artificial sequence
  chemically synthesized primer
agctgcatcaaccttcttcaca

SEQ ID NO: 9 - DNA, 14 bp, artificial sequence
  chemically synthesized probe
atgcagagaagccc

SEQ ID NO: 10 - DNA, 25 bp, artificial sequence
  chemically synthesized primer
gaggagagatgtttgtcttcaagga

SEQ ID NO: 11 - DNA, 24 bp, artificial sequence
  chemically synthesized primer
gggtatccatccatcacttggtta

SEQ ID NO: 12 - DNA, 16 bp, artificial sequence
  chemically synthesized probe
tcctcacccgccagag

SEQ ID NO: 13 - DNA, 20 bp, artificial sequence
  chemically synthesized primer
cacgaagtatccaagagctt

SEQ ID NO: 14 - DNA, 20 bp, artificial sequence
  chemically synthesized primer
catgctcttcagctttttac

SEQ ID NO: 15 - DNA, 20 bp, artificial sequence
  chemically synthesized primer
agagagccagacggaggaag

SEQ ID NO: 16 - DNA, 20 bp, artificial sequence
  chemically synthesized primer
gtcacactggtcactcctac

SEQ ID NO: 17 - protein, 470 aa, human
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDLHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 18 - protein, 6 aa, artificial sequence
  matrix metalloproteinase-12 substrate polypeptide
AMFLEA

SEQ ID NO: 19 - protein, 99 aa, human
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 20 - protein, 99 aa, human; identical to SEQ ID NO: 19